(19) United States
(12) Patent Application Publication
(10) Pub. No.: US 2021/0068961 A1
(43) Pub. Date: Mar. 11, 2021

(54) HARD-TISSUE IMPLANT COMPRISING A SHAFT, A SURFACE, PILLARS FOR CONTACTING A HARD TISSUE, SLOTS TO BE OCCUPIED BY THE HARD TISSUE, AND A THREAD DISPOSED HELICALLY ALONG THE SHAFT

(71) Applicant: GARY A. ZWICK, TRUSTEE OF THE EVEREST TRUST UTA APRIL 20, 2017, Cleveland, OH (US)

(72) Inventors: George J. PICHA, Brecksville, OH (US); James PRICE, Stow, OH (US); Gregory CAUSEY, Erie, CO (US)

(21) Appl. No.: 17/017,154

(22) Filed: Sep. 10, 2020

Related U.S. Application Data
(60) Provisional application No. 62/898,757, filed on Sep. 11, 2019.

(51) Int. Cl. A61F 2/30; A61F 2/42

(52) U.S. Cl. CPC A61F 2/30771; A61F 2/4225; A61F 2/4261; A61F 2002/30069; A61F 2002/30622; A61F 2002/30891; A61F 2002/30405; A61F 2002/4243

(57) ABSTRACT

A hard-tissue implant is provided. The implant includes a shaft, a surface of the shaft, pillars for contacting a hard tissue, slots to be occupied by the hard tissue, and a thread disposed helically along the shaft, extending radially from the shaft, and having a plurality of grooves oriented transversely with respect to the thread that define a series of thread segments and thread gaps along the thread. The implant has a Young's modulus of elasticity of at least 3 GPa and a ratio of (i) the sum of the volumes of the slots and the thread gaps to (ii) the sum of the volumes of the pillars and the thread segments and the volumes of the slots and the thread gaps of 0.40:1 to 0.90:1. Also provided is a method of use of the implant for fusion of two or more bones in an individual in need thereof.

### FIELD OF THE INVENTION

The invention relates to hard-tissue implants, and more particularly to hard-tissue implants comprising a shaft, a surface, pillars for contacting a hard tissue, slots to be occupied by the hard tissue, and a thread disposed helically along the shaft.

### BACKGROUND OF THE INVENTION

According to the Centers for Disease Control and Prevention, in 2015, 15 million adults in the United States reported severe joint pain due to arthritis. Joint fusion surgery, also termed arthrodesis, is a treatment for severe arthritis. Joint fusion surgery involves fusing two or more bones at a joint, which converts a stiff, painful joint into a stiff, non-painful joint.

Severe arthritis of the foot and ankle can make it difficult to walk and perform daily activities. Severe arthritis of the hand and wrist can be similarly debilitating. This can be due to osteoarthritis, in which cartilage in joints wears away, rheumatoid arthritis, in which the immune system attacks synovium covering joints, and/or posttraumatic arthritis, in which arthritis develops after injury.

Conventional approaches for joint fusion surgeries of the foot, ankle, hand, and wrist involve removal of damaged cartilage at a joint, followed by use of pins, plates and screws, or rods to fix the joint in place. Following the surgery, the bones at the joint gradually fuse by growing together.

Although arthrodesis is typically successful, complications can occur. In some cases, the joint does not fuse, and the hardware can break. Further surgeries may be needed, but repeated fusions are less likely to be successful. Also, some patients have problems with wound healing. In addition, loss of motion in one joint can cause adjacent joints to bear more stress, which can ultimately lead to arthritis in the adjacent joints.

Considering alternatives to pins, plates and screws, and rods for fixing joints, conventional hard-tissue implants include implants designed to promote in-growth of hard tissue based on forming a tissue/implant interface in which the implant forms a continuous phase and the tissue forms a discontinuous phase, e.g. based on the implant having a concave and/or porous surface into which the hard tissue can grow, and designed to have add-on surface modifications, e.g. modifications added based on sintering.

For example, Van Kampen et al., U.S. Pat. No. 4,608,052, discloses an implant for use in a human body having an integral attachment surface adapted to permit ingrowth of living tissue. The implant surface is defined by a multiplicity of adjacent, generally concave surface parts having intersecting, generally aligned rims defining an inner attachment surface portion and by a multiplicity of spaced posts projecting from the inner attachment surface. Van Kampen also discloses that implants have been provided with porous surfaces, as described in U.S. Pat. Nos. 3,605,123, 3,808,606, and 3,855,638.

Also for example, J. D. Bobyn et al, 150 Clinical Orthopaedics & Related Research 263 (1980), discloses that a pore size range of approximately 50 to 400 μm provided an optimal or maximal fixation strength (17 MPa) in the shortest time period (8 weeks) with regard to cobalt-base alloy implants with powder-made porous surfaces. Specifically, implants were fabricated based on coating cylindrical rods of cast cobalt-base alloy with cobalt base alloy powder in four particle size ranges. The particle size ranges were as follows: 25 to 45 μm; 45 to 150 μm; 150 to 300 μm; and 300 to 840 μm. The corresponding pore size ranges of the particles were as follows: 20 to 50 μm; 50 to 200 μm; 200 to 400 μm; and 400 to 800 μm, respectively. The particles were then bonded to the rods based on sintering. All implants were manufactured to have a maximal diameter of 4.5 mm and a length of 9.0 mm. The implants were surgically inserted into holes in dog femurs and bone ingrowth was allowed to proceed. After varying periods of time (4, 8, or 12 weeks), the maximum force required to dislodge the implants was determined. Implants with a pore size lower than 50 μm yielded relatively low fixation strengths at all time points, while implants with a pore size higher than 400 μm exhibited relatively high scatter with regard to fixation strengths, thus indicating that a pore size range of approximately 50 to 400 μm provided an optimal or maximal fixation strength.

Conventional hard-tissue implants also include implants having surface texturing, e.g. raised portions and indented portions, barbs, and/or pillars, to promote an interference fit between the implants and adjacent bone, to make it difficult to withdraw the implants from hard tissue, or to more effectively mechanically anchor at an early date or affix into adjoining hard tissue.

For example, Tuke et al., U.K. Pat. Appl. No. GB2181354A, discloses an orthopedic implant having at least one surface area, integral with the adjacent portion of the implant and adapted in use to contact bone. The surface area has a finely patterned conformation composed of a plurality of raised portions separated from each other by indented portions. The indented portions are of a width and depth to allow bone penetration thereinto in use to promote an interference fit between the implant and adjacent bone in the region of the patterned area.

Also for example, Amrich et al., U.S. Pat. No. 7,018,418, discloses implants having a textured surface with microrecesses such that the outer surface overhangs the microrecesses. In one embodiment, unidirectional barbs are produced in the surface that can be inserted into bone or tissue. The directional orientation of the barbs is intended to make it difficult to withdraw from the bone or tissue.

Also for example, Picha, U.S. Pat. No. 7,556,648, discloses a spinal implant, i.e. an implant for use in fusing and stabilizing adjoining spinal vertebrae, including a hollow, generally tubular shell having an exterior lateral surface, a leading end, and a trailing end. The exterior surface includes a plurality of pillars arranged in a non-helical array. Each pillar has a height of 100 to 4,500 μm and a lateral dimension at the widest point of 100 to 4,500 μm. The exterior surface also has a plurality of holes therethrough to permit bone ingrowth therethrough.

Unfortunately, interfaces of hard tissue and hard-tissue implants in which the hard tissue is in a discontinuous phase may be susceptible to stress shielding, resulting in resorption of affected hard tissue, e.g. bone resorption, over time. Also, addition of surface texturing to implants by sintering can result in the surface texturing occupying an excessive volume of corresponding hard tissue/implant interfaces, leaving insufficient space for hard tissue. In addition, spinal implants are designed to perform under conditions relevant to spine, i.e. compression, rotational shear, and vertical shear, with the compression being essentially constant, the rotational shear being intermittent, and the vertical shear being rare, rather than conditions relevant to other hard tissues such as long bone, maxillary bone, mandibular bone, and membranous bone, i.e. load bearing conditions, including compression and tension, varying across the hard tissue and across time, and intermittent rotational and vertical shear.

Picha et al., U.S. Pat. No. 8,771,354, discloses hard-tissue implants including a bulk implant, a face, pillars, and slots. The hard-tissue implant has a Young's modulus of elasticity of at least 10 GPa, has a ratio of (i) the sum of the volumes of the slots to (ii) the sum of the volumes of the pillars and the volumes of the slots of 0.40:1 to 0.90:1, does not comprise any part that is hollow, and does not comprise any non-pillar part extending to or beyond the distal ends of any of the pillars. The hard-tissue implants can provide immediate load transfer upon implantation and prevent stress shielding over time, thus promoting hard-tissue remodeling and growth at the site of implantation. The interface can have a continuous phase corresponding to the hard tissue and a discontinuous phase corresponding to the hard-tissue implant.

There remains a need for hard-tissue implants that address the issues discussed above regarding fixing joints of the foot, ankle, hand, and wrist, and that provide improvements. The hard-tissue implants disclosed herein are such implants.

### BRIEF SUMMARY OF THE INVENTION

A hard-tissue implant is provided. The hard-tissue implant comprises:
(a) a shaft having a top end and a bottom end, the shaft extending between the top end and the bottom end;
(b) a surface of the shaft extending from the top end to the bottom end;
(c) pillars for contacting a hard tissue, the pillars being distributed on the surface across an area of at least 50 mm², and extending distally therefrom, and each pillar being integral to the shaft, having a distal end, having a transverse area of (100×100) to (2,000×2,000) μm², and having a height of 100 to 2,000 μm;
(d) slots to be occupied by the hard tissue, the slots being defined by the pillars and each slot having a width of 100 to 2,000 μm as measured along the shortest distance between adjacent pillars; and
(e) a thread disposed helically along the shaft, extending radially from the shaft, and having a plurality of grooves oriented transversely with respect to the thread that define a series of thread segments and thread gaps along the thread.

The implant has a Young's modulus of elasticity of at least 3 GPa and a ratio of
(i) the sum of the volumes of the slots and the thread gaps to (ii) the sum of the volumes of the pillars and the thread segments and the volumes of the slots and the thread gaps of 0.40:1 to 0.90:1.

In some embodiments, the implant is made of one or more materials selected from implantable-grade polyaryletherketone that is essentially unfilled, implantable-grade polyetheretherketone, implantable-grade polyetherketoneketone, titanium, stainless steel, cobalt-chromium alloy, titanium alloy, Ti-6A1-4V titanium alloy, Ti-6A1-7Nb titanium alloy, ceramic material, silicon nitride (Si3N4), implantable-grade composite material, implantable-grade polyaryletherketone with filler, implantable-grade polyetheretherketone with filler, implantable-grade polyetheretherketone with carbon fiber, or implantable-grade polyetheretherketone with hydroxyapatite.

In some embodiments, the implant is made of one or more hard tissues selected from human hard tissue, animal hard tissue, autologous hard tissue, allogenic hard tissue, xenogeneic hard tissue, human cartilage, animal cartilage, human bone, animal bone, cadaver bone, or cortical allograft.

In some embodiments, the implant is made of one or more materials selected from resin for rapid prototyping, SOMOS (R) NanoTool non-crystalline composite material, SOMOS (R) 9120 liquid photopolymer, SOMOS (R) WaterShed XC 11122 resin, ACCURA (R) XTREME (TM) White 200 plastic, or ACCURA (R) 60) plastic.

In some embodiments, the shaft is straight.

In some embodiments, the shaft is tapered toward the bottom end.

In some embodiments, the shaft has a top end aperture located at the top end of the shaft.

In some embodiments, the pillars extend in a uniform direction. Also, in some embodiments, the pillars are perpendicular to the surface of the shaft. Also, in some embodiments, the pillars are angled toward the top end.

In some embodiments, the transverse area of each pillar is (250×250) μm² to (1,000×1,000) μm².

In some embodiments, the height of each pillar is 200 to 900 μm.

In some embodiments, one or more of the pillars have dimensions that differ from those of other pillars, such that the transverse areas and/or heights, and thus volumes, of the one or more pillars differ from those of the other pillars.

In some embodiments, the width of each slot is 200 to 1,000 μm.

In some embodiments, the thread has a thread height of 100 μm to 5,000 μm.

In some embodiments, the thread segments have a thread segment width, measured as an arcuate length with respect to the shaft, of 100 μm to 5,000 μm.

In some embodiments, the thread gaps have a thread gap width, measured as an arcuate length with respect to the shaft, of 100 μm to 5,000 μm.

In some embodiments, the shaft further comprises a non-threaded shaft portion between the head and the at least one thread.

In some embodiments, the shaft has a shaft diameter at a widest portion of the shaft and a shaft length from the top end to the bottom end, and the implant has a ratio of the shaft length to the shaft diameter of 2.0 to 10. In some embodiments, the shaft has a shaft diameter of 3 to 20 mm at a widest portion of the shaft. In some embodiments, the shaft has a shaft length of 6 to 40 mm from the top end to the bottom end.

In some embodiments, one or more of the shaft, the pillars, or the thread segments are non-porous. Also, in some embodiments, one or more of the shaft, the pillars, or the thread segments are porous.

In some embodiments, the implant further comprises a tool-engaging portion.

Also provided is a method of use of the hard-tissue implant for fusion of two or more bones in an individual in need thereof. The method comprises steps of:
(1) preparing a hole in at least a first bone and a second bone of the individual; and
(2) rotationally driving the implant into the hole, such that the implant contacts at least the first bone and the second bone and limits motion therebetween.

In some embodiments, the preparing of the hole comprises drilling a hole in at least the first bone and the second bone.

In some embodiments, the preparing of the hole comprises tapping the hole with a tapping device.

In some embodiments, the implant has an outer diameter between distal ends of pillars at a widest portion of the shaft, and the preparing of the hole comprises preparing the hole to have a hole diameter that is smaller than the outer diameter.

In some embodiments, the driving of the implant into the hole results in compression of at least the first bone and the second bone.

In some embodiments, the first bone comprises one or more metatarsal bones and the second bone comprises one or more cuneiform bones. In some of these embodiments, the driving of the implant into the hole limits motion of first and second metatarsals and medial and intermediate cuneiforms of the individual, corresponding to a 1-2 TMT fusion. Also, in some of these embodiments, the driving of the implant into the hole limits motion of second and third metatarsals and intermediate and lateral cuneiforms of the individual, corresponding to a 2-3 TMT fusion.

In some embodiments, the first bone comprises one or more navicular bones and the second bone comprises one or more cuneiform bones. In some of these embodiments, the driving of the implant into the hole limits motion of first and second navicular and medial and intermediate cuneiforms of the individual, corresponding to a 1-2 NC fusion. Also, in some of these embodiments, the driving of the implant into the hole limits motion of second and third navicular and intermediate and lateral cuneiforms of the individual, corresponding to a 2-3 NC fusion.

In some embodiments, the first bone comprises talus and the second bone comprises calcaneus.

In some embodiments, the first bone comprises a proximal phalanx of hand and the second bone comprises a middle phalanx of hand.

In some embodiments, the first bone comprises a middle phalanx of hand and the second bone comprises a distal phalanx of hand.

In some embodiments, the first bone comprises a first wrist bone and the second bone comprises a second wrist bone.

In some embodiments, the method further comprises:
(1) preparing another hole in at least the first bone and the second bone of the individual; and
(2) rotationally driving another of the implant into the hole, such that the other implant also contacts at least the first bone and the second bone and limits motion therebetween.

In some embodiments, the method does not comprise using plates or screws to limit motion between the first bone and the second bone.

### BRIEF DESCRIPTION OF THE DRAWINGS

These and other features, aspects, and advantages of the present disclosure are better understood when the following detailed description is read with reference to the accompanying drawings, in which:

FIG. 1 is a first perspective view of a first embodiment of a hard-tissue implant as disclosed herein;

FIG. 2 is a second perspective view of the implant of FIG. 1;

FIG. 3 is a first side view of the implant of FIG. 1;

FIG. 4 is a second side view of the implant of FIG. 1;

FIG. 5 is a top view of the implant of FIG. 1;

FIG. 6 is a bottom view of the implant of FIG. 1;

FIG. 7 is a sectional view of the implant of FIG. 6;

FIG. 8 is a first perspective view of a second embodiment of a hard-tissue implant as disclosed herein;

FIG. 9 is a second perspective view of the implant of FIG. 8;

FIG. 10 is a first side view of the implant of FIG. 8;

FIG. 11 is a second side view of the implant of FIG. 8;

FIG. 12 is a top view of the implant of FIG. 8;

FIG. 13 is a bottom view of the implant of FIG. 8;

FIG. 14 is a sectional view of the implant of FIG. 10;

FIG. 15 is a first perspective view of a third embodiment of a hard-tissue implant as disclosed herein;

FIG. 16 is a second perspective view of the implant of FIG. 15;

FIG. 17 is a first side view of the implant of FIG. 15;

FIG. 18 is a second side view of the implant of FIG. 15;

FIG. 19 is a top view of the implant of FIG. 15;

FIG. 20 is a bottom view of the implant of FIG. 15;

FIG. 21 is a sectional view of the implant of FIG. 17;

FIG. 22 is a first perspective view of a fourth embodiment of a hard-tissue implant as disclosed herein;

FIG. 23 is a second perspective view of the implant of FIG. 22;

FIG. 24 is a first side view of the implant of FIG. 22;

FIG. 25 is a second side view of the implant of FIG. 22;

FIG. 26 is a top view of the implant of FIG. 22;

FIG. 27 is a bottom view of the implant of FIG. 22;

FIG. 28 is a sectional view of the implant of FIG. 24;

FIG. 29 is a first perspective view of a fifth embodiment of a hard-tissue implant as disclosed herein;

FIG. 30 is a second perspective view of the implant of FIG. 29;

FIG. 31 is a first side view of the implant of FIG. 29;

FIG. 32 is a second side view of the implant of FIG. 29;

FIG. 33 is a top view of the implant of FIG. 29;

FIG. 34 is a bottom view of the implant of FIG. 29;

FIG. 35 is a sectional view of the implant of FIG. 31;

FIG. 36 is a first perspective view of a sixth embodiment of a hard-tissue implant as disclosed herein;

FIG. 37 is a second perspective view of the implant of FIG. 36;

FIG. 38 is a first side view of the implant of FIG. 36;

FIG. 39 is a second side view of the implant of FIG. 36;

FIG. 40 is a top view of the implant of FIG. 36;

FIG. 41 is a bottom view of the implant of FIG. 36;

FIG. 42 is a sectional view of the implant of FIG. 38;

FIG. 43 is a first perspective view of a first embodiment of another hard-tissue implant as disclosed herein;

FIG. 44 is a second perspective view of the implant of FIG. 43;

FIG. 45 is a first side view of the implant of FIG. 43;

FIG. 46 is a second side view of the implant of FIG. 43;

FIG. 47 is a top view of the implant of FIG. 43;

FIG. 48 is a bottom view of the implant of FIG. 43;

FIG. 49 is a sectional view of the implant of FIG. 48;

FIG. 50 is a first perspective view of a second embodiment of another hard-tissue implant as disclosed herein;

FIG. 51 is a second perspective view of the implant of FIG. 50;

FIG. 52 is a first side view of the implant of FIG. 50;

FIG. 53 is a second side view of the implant of FIG. 50;

FIG. 54 is a top view of the implant of FIG. 50;

FIG. 55 is a bottom view of the implant of FIG. 50; and

FIG. 56 is a sectional view of the implant of FIG. 50.

### DETAILED DESCRIPTION

As set forth in the figures, example hard-tissue implants are provided. The hard-tissue implants provide advantages, including for example that the hard-tissue implants can promote hard-tissue remodeling and growth of the hard tissue at the site of implantation and that the interface of the hard-tissue implants and the hard tissue can withstand substantial yield/elongation and load before failure. Without wishing to be bound by theory, it is believed that these advantages are based on properties of the hard-tissue implants and the interface resulting from implantation thereof, and that the hard-tissue implants can be particularly effective in joint fusion based on these properties and the resulting interface.

This is because the interface can have a continuous phase corresponding to the hard tissue and a discontinuous phase corresponding to the hard-tissue implant. The hard tissue can also make up at least 40% of the volume of the interface, and the product of the Young's modulus of elasticity of the hard tissue and the volume of the tissue and the product of the Young's modulus of elasticity of the implant and the volume of the pillars and the thread segments of the implant can be well matched. Thus, the interface can exhibit mechanical properties similar to those of the bulk hard tissue adjacent to the interface, in this case corresponding to two or more bones at a joint to be fused. The thread segments can be used for guiding the hard-tissue implant into threads of a hole in the two or more bones during rotational driving of the hard-tissue implant into the hole, and for removing small amounts of bone material during the driving by tapping the hole to have threads that have an inner diameter slightly smaller than an outer diameter of the implant including the thread segments. Also, the pillars may be rotationally driven into the hard-tissue during implantation, potentially eliminating micro-motion and migration of the implant over time, accommodating torque, and/or eliminating the need for adhesives such as cement or grout to hold the implant in place. In addition, the hard-tissue implants may promote rich vascularization of the hard tissue of the interface, enhancing wound healing, providing nutritional support, accelerating healing, remodeling, and integration of the hard tissue, and limiting the potential for infection of the hard tissue. Rapid or immediate integration of the hard tissue into the space between the pillars and thread segments of the hard-tissue implant may also prevent detrimental cellular reactions at the interface, such as formation of fibrous tissue, seroma, or thrombosis.

It is believed that implantation of the hard-tissue implant will result in the pillars and the thread segments contacting the hard tissue. In some cases the pillars and/or thread segments may initially penetrate the hard tissue, e.g. partially or completely, upon implantation of the hard-tissue implant. In such cases, the hard-tissue implants can provide immediate load transfer upon implantation and prevent stress shielding over time, thus promoting hard-tissue remodeling and growth at the site of implantation. Alternatively or additionally, in some cases the pillars and/or thread segments may penetrate the hard tissue later, under physiological loading. Also alternatively or additionally, over time the hard tissue may grow in and around the pillars and thread segments, thus occupying slots between the pillars and thread gaps between the thread segments, e.g. during healing.

The interface resulting from implantation of the hard-tissue implant into the hard tissue will be, or can become, an interface that is continuous with respect to the hard tissue and discontinuous with respect to the hard-tissue implant, across an area of the surface of the hard-tissue implant from which the pillars and the thread segments extend. Such an interface will further exhibit properties similar to those of the bulk hard tissue adjacent to the interface, e.g. high resilience to load. It is believed that such an interface will be particularly effective for joint fusion.

As used herein, the term “hard-tissue implant” means an implant suitable implantation in a hard tissue. Exemplary hard-tissue implants include implants for joint fusion. Exemplary hard tissues suitable for implantation of the hard-tissue implants include metatarsal bones, cuneiform bones, navicular bones, talus, calcaneus, proximal, middle, and distal phalanges of hand, and wrist bones. Exemplary joint fusions suitable for the hard-tissue implants include fusion of first and second metatarsals and medial and intermediate cuneiforms (also termed “1-2 TMT fusion”), fusion of second and third metatarsals and intermediate and lateral cuneiforms (also termed “2-3 TMT fusion”), fusion of first and second navicular and medial and intermediate cuneiforms (also termed “1-2 NC fusion”), fusion of second and third navicular and intermediate and lateral cuneiforms (also termed 2-3 NC fusion), fusion of talus and calcaneus, fusion of proximal and middle phalanges of hand, fusion of middle and distal phalanges of hand, and fusion of wrist bones.

As used herein, the term “pillar” means a projection that extends distally from a surface of an implant, that is not in direct physical contact with any other pillars or other parts of the implant other than the surface, and that is for contacting a hard tissue. Because a pillar is not in direct physical contact with any other pillars or other parts of the implant other than the surface, upon implantation no pillar forms a continuous phase within the resulting interface of the hard tissue and the hard-tissue implant.

A pillar can have a transverse area, i.e. an area of a cross-section taken relative to a vertical axis along which the pillar extends distally from the surface of the implant, of, for example, (i) (100 μm×100 μm) to (2,000 μm×2,000 μm), i.e. 1.0×10⁴ μm² to 4.0×10⁶ μm², (ii) (200 μm×200 μm) to (1,000 μm×1,000 μm), i.e. 4.0×10⁴ μm² to 1.0×10⁶ μm², (iii) (250 μm×250 μm) to (1,000 μm×1,000 μm), i.e. 6.3×10⁴ μm² to 1.0×10⁶ μm², (iv) (300 μm×300 μm) to (500 μm×500 μm), i.e. 9×10⁴ μμm² to 2.5×10⁵ μm², (v) (350 μm×350 μm) to (450 μm×450 μm), i.e. 1.2×10⁵ μm² to 2.0×10⁵ μm², or (vi) (395 μm×395 μm) to (405 μm×405 μm), i.e. 1.6×10⁵ μm². Of note, the expression of transverse areas of pillars as squares of linear dimensions, e.g. (100 μm×100 μm), here and throughout this application, is for purposes of convenience only and is not intended to limit any pillars so described to square shapes, square transverse areas, or square cross-sections.

A pillar can have a pillar height, i.e. the height of the pillar from a surface of the implant to the distal end of the pillar, of, for example, 100 to 2,000 μm, 200 to 900 μm, 300 to 800 μm, or 400 to 600 μm.

A pillar can have a volume, i.e. product of pillar transverse area and pillar height, of, for example (100 μm×100 μm×100 μm) to (2,000 μm×2,000 μm×2,000 μm), i.e. 1.0×10⁶ μm³ to 8×10⁹ μm³, among other volumes.

A pillar can have, as seen from a top view, a square shape, a rectangular shape, a herringbone shape, a circular shape, or an oval shape, respectively, or alternatively can have other polygonal, curvilinear, or variable shapes.

As used herein, the term “slot” means the spaces between the pillars. Accordingly, the pillars define the slots. The slots can have a slot height as defined by the pillars, of, for example, 100 to 2,000 μm, 200 to 900 μm, 300 to 800 μm, or 400 to 600 μm, among others. The slots can have a slot width as measured along the shortest distance between adjacent pillars of, for example, 100 to 2,000 μm, 150 to 1,000 μm, 200 to 700 μm, or 300 to 500 μm, among others. The slots have a volume corresponding to the volume of the space between the pillars.

As used herein, the term “pore” refers to a void space of less than 1,000 um in size, i.e. having a diameter of less than 1,000 μm, on or below a surface, e.g. the surface of an implant. Pores can occur in a material naturally, e.g. based on a natural porosity of the material, or can be introduced, e.g. by chemical or physical treatment. Pores can be continuous with respect to each other, based on being interconnected with each other below a surface, or pores can be discontinuous, based on not being interconnected with each other below a surface. Pores can be sufficiently large to allow for migration and proliferation of osteoblasts and mesenchymal cells. Accordingly, for example, a porous surface is a surface that includes void spaces of less than 1,000 μm in size in the surface, whereas a non-porous surface is a surface that does not include such a void space.

As used herein, the term “interface” includes the product of implantation wherein the pillars and thread segments of the hard-tissue implant are contacting a hard tissue and the slots and thread gaps of the implant are occupied, partially or completely, by the hard tissue.

In some examples, e.g. immediately after implanting the implant with at least some penetration of the pillars and/or thread segments into the hard tissue and/or after at least some remodeling and growth of the hard tissue to partially fill in space between the implant and the hard tissue, the pillars and/or thread segments are contacting the hard tissue (e.g. at distal ends of the pillars), and the slots and/or thread gaps are partially occupied by the hard tissue. In other examples, e.g. immediately after implanting the implant with extensive penetration of the pillars and thread segments into the hard-tissue and/or after extensive remodeling and growth of the hard tissue to fill in all space between the implant and the hard tissue, the pillars and thread segments are contacting the hard tissue (e.g. at distal ends and lateral surfaces of the pillars and along surfaces of the thread segments), and the slots and thread gaps are completely occupied by the hard tissue. In other examples the pillars and/or thread segments contact the hard tissue over time, based on remodeling and growth of hard tissue in and around the pillars and thread segments, e.g. during healing.

As used herein, the term “continuous,” when used for example in reference to the hard-tissue of an interface, means that the hard tissue forms a single continuous phase, extending throughout and across the interface to each boundary of the interface. As used herein, the term “discontinuous,” when used for example in reference to the implant of an interface, means that the implant does not form such a single continuous phase.

Hard-Tissue Implant

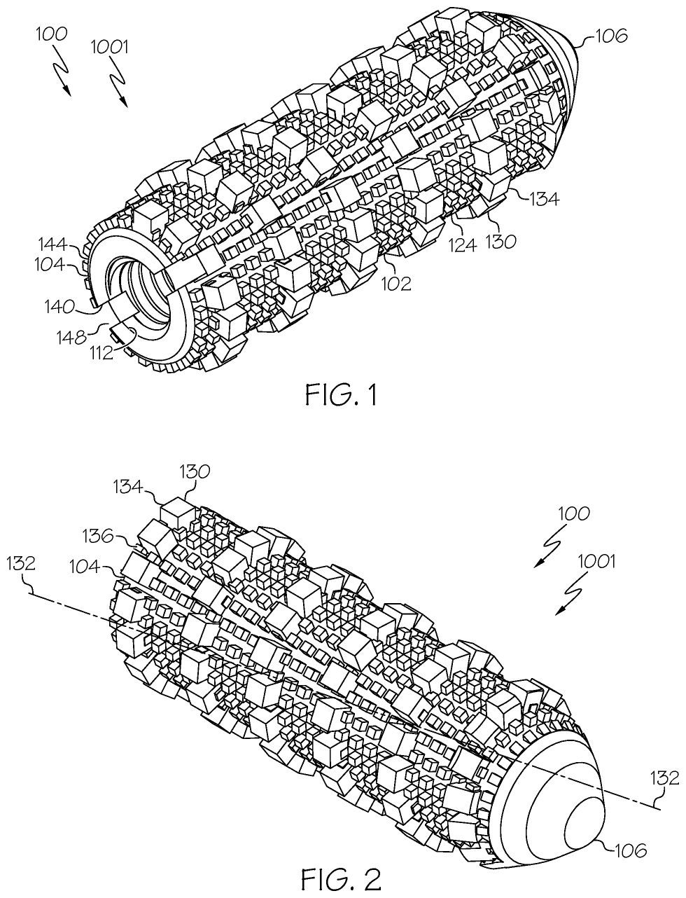

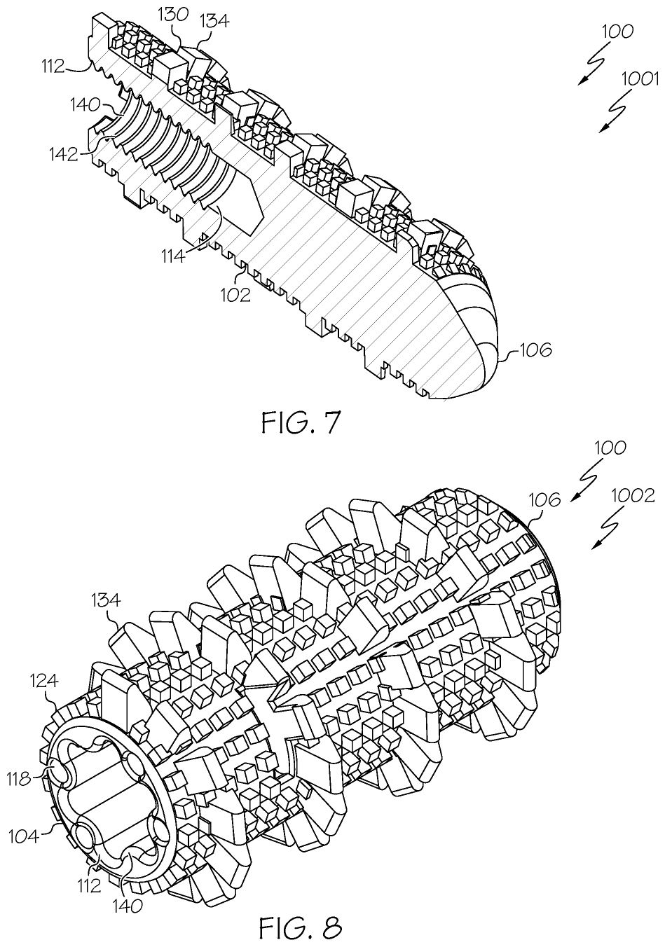

Considering the features of the hard-tissue implant in more detail, FIGS. 1-7 illustrate a first embodiment 1001 of a hard-tissue implant 100.

The hard-tissue implant 100 can be made from a material having a Young's modulus of elasticity, i.e. a tensile modulus of elasticity, of at least 3 GPa, as measured at 21° C. The hard-tissue implant 100 can be made, for example, from one or more materials such as implantable-grade polyaryletherketone that is essentially unfilled (such as implantable-grade polyetheretherketone or implantable-grade polyetherketoneketone), titanium, stainless steel, cobalt-chromium alloy, titanium alloy (such as Ti-6Al-4V titanium alloy or Ti-6Al-7Nb titanium alloy), ceramic material (such as silicon nitride (Si3N4)), or implantable-grade composite material (such as implantable-grade polyaryletherketone with filler, implantable-grade polyetheretherketone with filler, implantable-grade polyetheretherketone with carbon fiber, or implantable-grade polyetheretherketone with hydroxyapatite). Specific examples include (i) implantable-grade polyetheretherketone that is essentially unfilled, which has a Young's modulus of approximately 4 GPa, (ii) implantable-grade polyetheretherketone with filler, e.g. carbon-fiber-reinforced implantable-grade polyetheretherketone, which has a Young's modulus of elasticity of at least 18 GPa, (iii) titanium, which has a Young's modulus of elasticity of approximately 110 GPa, (iv) stainless steel, which has a Young's modulus of elasticity of approximately 200 GPa, (v) cobalt-chromium alloy, which has a Young's modulus of elasticity of greater than 200 GPa, or (vi) titanium alloy, which has a Young's modulus of elasticity of approximately 105-120 GPa, all as measured at 21° C. The hard-tissue implant 100 also can be made, for example, from one or more hard tissues such as a hard tissue obtained from a human or animal (such as autologous hard tissue, allogenic hard tissue, or xenogeneic hard tissue), human cartilage, animal cartilage, human bone, animal bone, cadaver bone, or cortical allograft. Such hard tissues obtained from a human or animal can have a Young's modulus of elasticity of, e.g. 4 to 18 GPa. Such hard tissues obtained from a human or animal can also be treated, in advance of implantation, to decrease or eliminate the capacity of the hard tissue to elicit an immune response in an individual upon implantation into the individual. The hard-tissue implant 100 also can be made, for example, from one or more materials such as resin for rapid prototyping, SOMOS (R) NanoTool non-crystalline composite material, SOMOS (R) 9120 liquid photopolymer, SOMOS (R) WaterShed XC 11122 resin, ACCURA (R) XTREME (TM) White 200 plastic, or ACCURA (R) 60) plastic. The hard-tissue implant 100 also can be made from further combinations of the above-noted materials and/or hard tissues. Accordingly, the hard-tissue implant 100 has a Young's modulus of elasticity of at least 3 GPa, for example 18 to 230 GPa, 18 to 25 GPa, 100 to 110 GPa, 190 to 210 GPa, 200 to 230 GPa, 105 to 120 GPa, or 4 to 18 GPa.

As shown in FIGS. 1-7, the hard-tissue implant 100 comprises a shaft 102 having a top end 104 and a bottom end 106. The shaft 102 extends between the top end 104 and the bottom end 106.

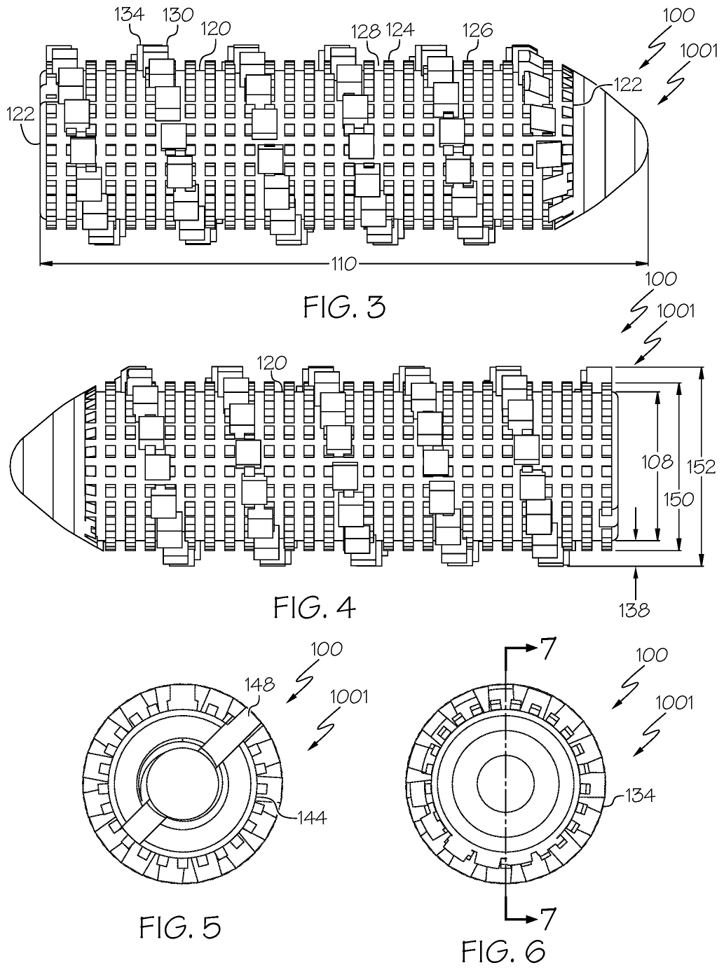

The shaft 102 forms the core of the hard-tissue implant 100 and can have a generally cylindrical shape, although other shapes, e.g. conical shapes, or frustoconical shapes, may be used in further examples. As shown in FIG. 3 and FIG. 4, in some examples the shaft 102 has a shaft diameter 108 at a widest portion of the shaft 102 and a shaft length 110 from the top end 104 to the bottom end 106, and the hard-tissue implant 100 has a ratio of the shaft length 110 to the shaft diameter 108 of 2.0 to 10. In some examples the shaft 102 has a shaft diameter 108 of 3 to 20 mm at a widest portion of the shaft 102. In some examples the shaft 102 has a shaft length 110 of 6 to 40 mm from the top end 104 to the bottom end 106.

The shaft 102 can be made from one or more of the materials or hard tissues noted above with respect to the hard-tissue implant 100, e.g. one or more materials such as implantable-grade polyaryletherketone that is essentially unfilled (such as implantable-grade polyetheretherketone or implantable-grade polyetherketoneketone), titanium, stainless steel, cobalt-chromium alloy, titanium alloy (such as Ti-6Al-4V titanium alloy or Ti-6Al-7Nb titanium alloy), ceramic material (such as silicon nitride (Si3N4)), or implantable-grade composite material (such as implantable-grade polyaryletherketone with filler, implantable-grade polyetheretherketone with filler, implantable-grade polyetheretherketone with carbon fiber, or implantable-grade polyetheretherketone with hydroxyapatite), or e.g. one or more hard tissues such as a hard tissue obtained from a human or animal (such as autologous hard tissue, allogenic hard tissue, or xenogeneic hard tissue), human cartilage, animal cartilage, human bone, animal bone, cadaver bone, or cortical allograft, or e.g. one or more materials such as resin for rapid prototyping, SOMOS (R) NanoTool non-crystalline composite material, SOMOS (R) 9120 liquid photopolymer, SOMOS (R) WaterShed XC 11122 resin, ACCURA (R) XTREME (TM) White 200 plastic, or ACCURA (R) 60) plastic.

The shaft 102 can be porous or non-porous. For example, the shaft 102 can include one or more surfaces that are porous, and/or can be made from one or more materials that are porous. Such porous surfaces can include pores having diameters of, e.g. 1 to 900 μm, 100 to 800 μm, or 200 to 600 μm. Also for example, the shaft 102 can include only surfaces that are non-porous, and/or can be made only from one or more materials that are non-porous.

As shown in FIGS. 1-7, in some examples the shaft 102 is straight. In some examples the shaft 102 is tapered toward the bottom end 106. In some examples the shaft 102 has a top end aperture 112 located at the top end 104 of the shaft 102. In some of these examples the shaft 102 has an internal passage 114 extending axially with respect to the shaft 102, from the top end aperture 112. In some examples the internal passage 114 extends through the shaft 102, from the top end aperture 112, and ends within the shaft 102. Accordingly, in some examples the hard-tissue implant 100 includes a blind hole. Also, in some examples the internal passage 114 extends through the shaft 102, from the top end aperture 112, to a bottom end aperture 116 located at the bottom end 106 of the shaft 102. Accordingly, in some examples the hard-tissue implant 100 is cannulated. The cannula can have a diameter of, for example, 1 to 3 mm diameter. Also, in some examples the internal passage 114 is sufficiently extensive that the shaft 102 is essentially hollow. In these examples the internal passage 114 may be supplemented with, for example, a material for osseointegration, an anti-bacterial material, and/or a medication. These features can facilitate implantation of the hard-tissue implant 100 into a site for implantation in a hard tissue, e.g. into a hole prepared in two or more bones, for example by providing a complementary fit between the hard-tissue implant 100 and the site for implantation, allowing easy insertion of the hard-tissue implant 100, and allowing use of a tool and/or guidewire for guiding the hard-tissue implant 100 during insertion. In some examples the shaft 102 comprises one or more channels 118 through which sutures can be passed. Sutures passed through the channels 118 can be used, for example, to tie or drawn down soft tissue to a bone surface or in a hole.

As shown in FIG. 3 and FIG. 4, the hard-tissue implant 100 also comprises a surface 120 of the shaft 102 extending from the top end 104 to the bottom end 106. The surface 120 is an exterior surface of the shaft 102.

The surface 120 can be defined by an edge 122. For example, the edge 122 can be a single continuous edge that defines the surface 120, e.g. an edge 122 at the top end 104 of the shaft 102, or an edge 122 at the bottom end 106 of the shaft 102. Also for example, the edge 122 can be two edges that are discontinuous with respect to each other that together define the surface 120, e.g. an edge 122 at the top end 104 of the shaft 102 and an edge 122 at the bottom end 106 of the shaft 102. The edge 122 can be sharp, although other rounded, angular, smooth, and/or irregular edges may be used in further examples.

The top end can have a top end surface that is, for example, flat, raised, or irregular, among other contours. The bottom end can have a shape that is bullet-shaped, blunt, pointed, conical, or frustoconical, among other shapes.

The surface 120 can be porous, e.g. including pores having diameters of, e.g. 1 to 900 μm, 100 to 800 μm, or 200 to 600 μm, or the surface 120 can be non-porous.

As shown in FIGS. 1-7, the hard-tissue implant 100 also comprises pillars 124 for contacting a hard tissue. The hard tissue can be selected from, for example, bones such as metatarsal bones, cuneiform bones, navicular bones, talus, calcaneus, proximal, middle, and distal phalanges of hand, and wrist bones among other hard-tissues. In some examples the pillars 124 may contact a hard tissue immediately upon implantation, e.g. based on extending distally from the surface of the shaft 102. In some examples the pillars 124 may contact a hard tissue over time after implantation, e.g. based on remodeling and growth of a hard tissue to come in contact with pillars 124 over time after implantation.

The pillars 124 are distributed on the surface 120 across an area of at least 50 mm². For example, the pillars 124 can be distributed in a regular pattern on the surface 120, across the area of the surface 120. In this regard, the pillars 124 can be distributed in even rows along the surface 120, and can be distributed along a given row uniformly with respect to the distances between the centers of the pillars 124 in the row. Also for example, the pillars 124 can also be distributed in other regular patterns, e.g. the pillars 124 can be distributed in rows that are even, but without the pillars 124 being distributed uniformly within rows, the pillars 124 in one row may be offset from the pillars 124 in adjacent rows, the pillars 124 may be arranged in a spiral pattern, etc. Also for example, the pillars 124 can be distributed on the surface in irregular patterns or randomly. For example, the pillars 124 can be distributed on the surface 120 such that the pillars 124 are packed more densely on one area of the surface 120 and less densely on another area of the surface 120.

The pillars 124 can be distributed on the surface 120 of the shaft 102 such that none of the pillars 124 are located at an edge 122 of the surface 120, i.e. the surface 120 can have a peripheral border that is not occupied by any pillars 124, resulting in the area of the surface 120 across which the pillars 124 are distributed being less than the total area of the surface 120. In other examples the pillars 124 can be distributed on the surface 120 such that at least some of the pillars 124 are located at an edge 122, e.g. the area of the surface 120 across which the pillars 124 are distributed can be equal to the total area of the surface 120.

The pillars 124 extend distally from the surface 120. In some examples all pillars 124 extend in a uniform direction. In some examples all pillars 124 extend distally at the same angle with respect to the first surface 120. Also for example, some pillars 124 may extend distally at a different angle and/or in a different direction relative to other pillars 124. In some examples the pillars 124 extend perpendicularly from the surface 120. This can simplify manufacturing of the hard-tissue implant 100. In some examples the pillars 124 are angled toward the top end 104 of the shaft 102. This can increase stability of the hard-tissue implant 100 following implantation in a hard tissue e.g. an implant 100 including pillars 124 angled this way can resist pull-out. In some examples the pillars 124 extend from the surface 120 at other angles and/or varying angles.

Each pillar 124 is integral to the shaft 102, i.e. the pillars 124 and the shaft 102 are made from the same starting material, rather than, for example, the pillars 124 being an add-on to the shaft 102. Like the shaft 102, the pillars 124 can be porous, e.g. including pores having diameters of, e.g. 1 to 900 100 to 800 or 200 to 600 or the pillars 124 can be non-porous.

Each pillar 124 has a distal end 126, corresponding to the distal-most portion of the pillar 124 relative to the surface 120 of the shaft 102. Each pillar 124 can have distal edges, corresponding to edges defining the distal end 126 of each pillar 124. Each pillar 124 can also have lateral edges, corresponding to edges of the lateral sides of each pillar 124. The distal edges and/or the lateral edges can be sharp, although other rounded, angular, smooth, and/or irregular edges may be used in further examples. The distal ends 126 can be flat, slanted, curved, or pointed, among other contours.

With respect to dimensions of the pillars 124, each pillar 124 has a transverse area, i.e. an area of a cross-section taken relative to the vertical axis along which the pillar 124 extends distally from the surface 120, of (100×100) to (2,000×2,000) μm². Each pillar 124 can have a transverse area of, for example, (200 μm×200 μm) to (1,000 μm×1,000 μm), (250 μm×250 μm) to (1,000 μm×1,000 μm), (300 μm×300 μm) to (500 μm×500 μm), (350 μm×350 μm) to (450 μm×450 μm), or (395 μm×395 μm) to (405 μm×405 μm). Each pillar 124 has a pillar height, i.e. the height of the pillar 124 from the surface 120 of the shaft 102 to the distal end 126 of the pillar 124, of 100 to 2,000 μm. Each pillar 124 can have a pillar height of, for example, 200 to 900 μm, 300 to 800 μm, or 400 to 600 μm. Each pillar 124 has a volume, i.e. product of pillar transverse area and pillar height, of, for example (100 μm×100 μm×100 μm) to (2,000 μm×2,000 μm×2,000 μm), i.e. 1.0×10⁶ μm³ to 8×10⁹ μm³, among other volumes. The pillars 124 extending from the surface 120 can, for example, all have identical dimensions, e.g. identical pillar transverse areas, pillars heights, and thus identical individual volumes. Alternatively, one or more pillars 124 can have dimensions that differ from those of other pillars 124, such that the pillar transverse areas and/or pillar heights, and thus volumes, of the one or more pillars 124 differ from those of the other pillars 124.

The pillars 124 can have, as seen from a top view, a square shape, a rectangular shape, a herringbone shape, a circular shape, or an oval shape, or alternatively can have other polygonal, curvilinear, or variable shapes. In some examples all pillars 124 can have the same shape, e.g. a square shape, a rectangular shape, a herringbone shape, a circular shape, or an oval shape, as seen from a top view. In some examples not all pillars 124 have the same shape as seen from a top view.

As shown in FIG. 3, the hard-tissue implant 100 also comprises slots 128 to be occupied by the hard tissue. For example, upon implantation of the hard-tissue implant 100 into a hard tissue, the hard tissue can immediately occupy all or part of the space corresponding to the slots 128. This can be accomplished, for example, by drilling a hole in two or more bones such that the hole has an inner diameter, not including threads added by tapping, slightly smaller than an outer diameter of the hard-tissue implant 100 including the pillars 124, then rotationally driving the hard-tissue implant 100 into the hole. Moreover, to the extent that the hard tissue does not, upon implantation, immediately occupy all of the space corresponding to slots 128, the hard tissue can eventually occupy all or part of the space corresponding to the slots 128 based on remodeling and/or growth of the hard tissue over time, e.g. during healing.

The slots 128 are defined by the pillars 124, i.e. the slots 128 are the spaces between the pillars 124. Accordingly, the slots 128 have a slot height as defined by the pillars 124, of, for example, 100 to 2,000 μm, 200 to 900 μm, 300 to 800 μm, or 400 to 600 μm. Each slot 128 has a width of 100 to 2,000 μm as measured along the shortest distance between adjacent pillars 124. The slot width can be, for example, 150 to 1,000 μm, 200 to 700 μm, or 300 to 500 μm. The slots 128 have a volume corresponding to the volume of the space between the pillars 124.

As shown in FIGS. 1-7, the hard-tissue implant 100 also comprises a thread 130 disposed helically along the shaft 102. The thread 130 extends radially from the shaft 102. The thread 130 has a plurality of grooves 132 oriented transversely with respect to the thread 130 that define a series of thread segments 134 and thread gaps 136 along the thread 130.

Like the pillars 124, the thread 130 is integral to the shaft 102, i.e. the thread 130 and the shaft 102 are made from the same starting material, rather than, for example, the thread 130 being an add-on to the shaft 102. Also, the thread segments 134 can be porous, e.g. including pores having diameters of, e.g. 1 to 900 μm, 100 to 800 μm, or 200 to 600 μm, or the thread segments 134 can be non-porous. Also, the thread segments 134 can be disposed on the surface 120 of the shaft 102 such that none of the thread segments 134 are located at an edge 122 of the surface 120, or such that at least some thread segments 134 are located at an edge 122.

In some examples the thread 130 has a thread height 100 μm to 5,000 μm. The thread height corresponds to the distance 138 between the surface 120 of the shaft 102 and a maximal diameter of the thread 130. The maximal diameter of the thread 130 can be defined by the thread 130 at a furthest point at which the thread 130 extends radially from the shaft 102. The thread height can vary along the hard-tissue implant 100, depending for example on dimensions of the shaft 102 and the thread 130 along the hard-tissue implant 100. For example, the thread height can vary along the hard-tissue implant 100 in accordance with loads that the hard-tissue implant 100 will need to carry and/or hard tissue with which the hard-tissue implant 100 will interface. In some examples, the thread 130 has a thread height of 120 μm to 4,000 μm, 150 μm to 3,000 μm, 200 μm to 2,000 μm, 250 μm to 1,500 μm, 300 μm to 1,200 μm, or about 1,000 μm.

In some examples the thread segments 134 have a thread segment width, measured as an arcuate length with respect to the shaft 102, of 100 μm to 5,000 μm. The thread segment width can be measured along the thread 130 at a furthest point at which the thread extends radially from the shaft 102. The arcuate length can be measured with respect to a center line along a major axis of the shaft 102. The thread segment width can vary along the hard-tissue implant 100, depending for example on dimensions of the shaft 102, the thread 130, and the plurality of grooves 132 along the hard-tissue implant 100. In some examples, the thread segments 134 have a thread segment width of 120 μm to 4,000 μm, 150 μm to 3,000 μm, 200 μm to 2,000 μm, 250 μm to 1,500 μm, 300 μm to 1,200 μm, or about 1,000 μm.

In some examples the thread gaps 136 have a thread gap width, measured as an arcuate length with respect to the shaft 102, of 100 μm to 5,000 μm. The thread gap width can be measured analogously to the thread segment width, along the thread 130 at a furthest point at which the thread 130 extends radially from the shaft 102. The arcuate length can be measured with respect to a center line along a major axis of the shaft 102. The thread gap width can vary along the hard-tissue implant 100, depending for example on dimensions of the shaft 102, the thread 130, and the plurality of grooves 132 along the hard-tissue implant 100. In some examples, the thread gaps 136 have a thread gap width of 120 μm to 4,000 μm, 150 μm to 1,000 μm, 200 μm to 800 μm, 250 μm to 600 μm, 300 μm to 500 μm, or about 400 μm.

The hard-tissue implant 100 can be made to have thread segments 134 having edge shapes suitable for a specific orthopedic application and/or hard tissue. For example, in some embodiments the thread segments 134 can have an edge shape at a maximal diameter of the thread segments 134, i.e. at a furthest point at which the thread segment 134 extends radially from the shaft 102, corresponding to an acute angle form, e.g. a sharp V-form. This may promote initial implantation of the hard-tissue implant 100 into a hard tissue. Also in some examples the thread segments 134 can have an edge shape at a maximal diameter of the thread segments 134 corresponding to a radiused form, e.g. a rounded form. This may avoid irritation to hard tissue during loading. Similarly, in some embodiments the thread segments 134 can have an edge shape at a position at which the thread segment 134 extends radially from the surface of shaft 102 corresponding to an acute angle form. Also in some embodiments the thread segments 134 can have an edge shape at this position corresponding to a radiused form. Additional edge shapes, e.g. a blunt form or an irregular form, among others, may also be used.

As noted, the hard-tissue implant 100 has a plurality of grooves 132, i.e. two or more grooves 132. In some examples, the plurality of grooves 132 comprises three or more grooves, e.g. 4-6, 7-10, 11-20, 20-40, or more than 40 grooves.

In some examples, the shaft 102 further comprises a non-threaded shaft portion between the top end 104 of the shaft 102 and the at least one thread 130.

The hard-tissue implant 100 has a ratio of (i) the sum of the volumes of the slots 128 and the thread gaps 136 to (ii) the sum of the volumes of the pillars 124 and the thread segments 134 and the volumes of the slots 128 and the thread gaps 136 of 0.40:1 to 0.90:1.

Without wishing to be bound by theory, it is believed that this ratio determines the approximate percentages of hard tissue and hard-tissue implant 100 that will occupy an interface following implantation of the hard-tissue implant 100, e.g. that upon inserting the hard-tissue implant 100 into the hard tissue, or upon remodeling and growth of the hard-tissue following implantation, that the hard tissue will occupy all or essentially all of the space corresponding to the slots 128 and thread gaps 136 of the hard-tissue implant 100. The interface includes (i) the pillars 124 and thread segments 134, (ii) the slots 128 and thread gaps 136, which, upon or following implantation, become occupied by hard tissue, (iii) any additional space between the surface 120 of the shaft 102 and a curved surface defined by the distal ends 126 of the pillars 124, e.g. the space between peripheral borders of the surface 120 at the top end 104 and the bottom end 106 of the shaft 102 that is not occupied by pillars 124 or thread segments 134 and the curved surface, which also becomes occupied by hard tissue, and (iv) any pores on the surface 120, the pillars 124, and/or the thread segments 134 which, depending on their size, may also become occupied by hard tissue. Accordingly, for example, a ratio as described of 0.40:1 would, following implantation of an hard-tissue implant 100 and subsequent remodeling and growth of hard tissue, wherein the hard-tissue implant 100 includes an edge 122 around the surface 120 and for which pillars 124 and/or thread segments 134 are located at the edge 122, result in an interface that includes by volume 40% hard tissue and 60% implant, and more particularly 60% pillars 124 and/or thread segments 134 of the hard-tissue implant 100. Similarly, a ratio as described of 0.40:1 would, following implantation of an hard-tissue implant 100 and subsequent remodeling and growth of hard tissue, wherein the hard-tissue implant 100 includes an edge 122 around the surface 120 and for which no pillars 124 or thread segments 134 are located at the edge 122, result in an interface that includes by volume more than 40% hard tissue and less than 60% implant, with the percentage of hard tissue increasing, and the percentage of hard-tissue implant 100 decreasing, with increasing distance between the peripheral-most pillars 124 and/or thread segments 134 and the edge 122 around the surface 120. By way of further examples, ratios of 0.51:1, 0.60:1, 0.70:1, 0.76:1, and 0.90:1, would result in interfaces that include, by volume, 51% hard tissue and 49% implant, 60% hard tissue and 40% implant, 70% hard tissue and 30% implant, 76% hard tissue and 24% implant, and 90% hard tissue and 10% implant, respectively, for a hard-tissue implant 100 wherein the hard-tissue implant 100 includes an edge 122 around the surface 120 and for which pillars 124 and/or thread segments 134 are located at the edge 122. Moreover, the percentage of hard tissue would increase, and the percentage of implant would decrease, with increasing distance between the peripheral-most pillars 124 and/or thread segments 134 and the edge 122 of the surface 120. It is believed that by achieving an interface that is at least 40% hard tissue, but that has a sufficient amount of the hard-tissue implant 100 to provide support and to keep the hard-tissue implant 100 from migrating, the interface will exhibit properties similar to those of the bulk hard tissue adjacent to the interface, e.g. high resilience to load.

As shown in FIG. 1 and FIG. 7, in some examples the hard-tissue implant 100 further comprises a tool-engaging portion 140. In some examples the tool-engaging portion 140 comprises a thread 142 located in an internal passage 114 of the shaft 102 for engaging a tool, e.g. a tool to drive the hard-tissue implant 100 into a hard tissue by rotation. For example, as noted above, in some examples the shaft 102 has a top end aperture 112 located at the top end 104 of the shaft 102 and an internal passage 114 extending axially with respect to the shaft 102, from the top end aperture 112. Also, in some examples the internal passage 114 extends through the shaft 102, from the top end aperture 112, and ends within the shaft 102. In some of these examples the tool-engaging portion 140 comprises the thread 142 located in the internal passage 114. Alternatively or additionally, in some examples the tool-engaging portion 140 comprises a head 144 including notches 148 located at the top end 104 of the shaft 102 for engaging a tool, e.g. a tool to rotationally drive the hard-tissue implant 100 into a hard tissue. Alternatively or additionally, in some examples the tool-engaging portion 140 comprises a head 144 including a recess 146 located at the top end 104 of the shaft 102 for engaging a tool, e.g. again a tool to rotationally drive the hard-tissue implant 100 into a hard tissue. Other tool-engaging portions 140 suitable for driving, pressing, or otherwise inserting the hard-tissue implant 100 into a hard tissue also can be used.

In some examples, the hard-tissue implant 100 further comprises one or more holes at or near the bottom end 106 of the shaft 102. The holes can be used for passing a suture. The suture can then be used for pulling the hard-tissue implant 100 into a hard tissue, e.g. into a hole in a hard tissue.

In some examples, the hard-tissue implant 100 further comprises a plate and screws. These can be used to secure the hard-tissue implant 100 to a hard tissue following implantation. In other examples, the hard-tissue implant 100 does not comprise a plate or screws, e.g. in cases for which the pillars 124 and the thread segments 134 secure the hard-tissue implant 100 to a hard tissue sufficiently.

In accordance with the first embodiment 1001 of a hard-tissue implant 100, as shown in FIGS. 1-7, the shaft 102 of the hard-tissue implant 100 comprises a top end aperture 112. The shaft 102 has an internal passage 114 extending axially with respect to the shaft 102, from the top end aperture 112. The internal passage 114 extends through the shaft 102, from the top end aperture 112, and ends within the shaft 102. The hard-tissue implant 100 further comprises a tool-engaging portion 140, comprising a thread 142 located in the internal passage 114 of the shaft 102, and a head 144 including notches 148 located at the top end 104 of the shaft 102.

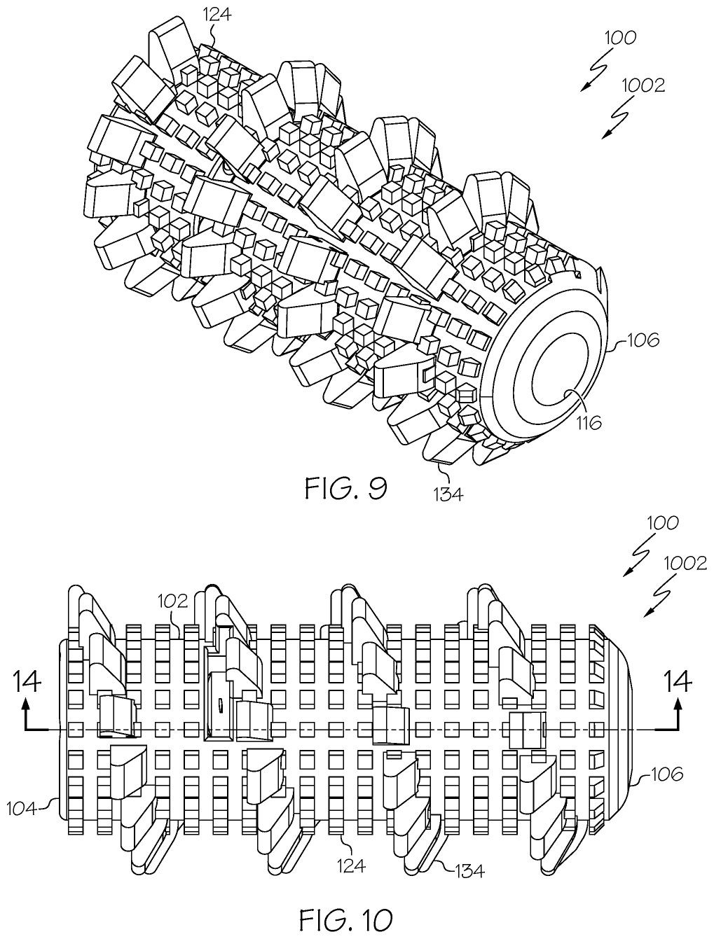

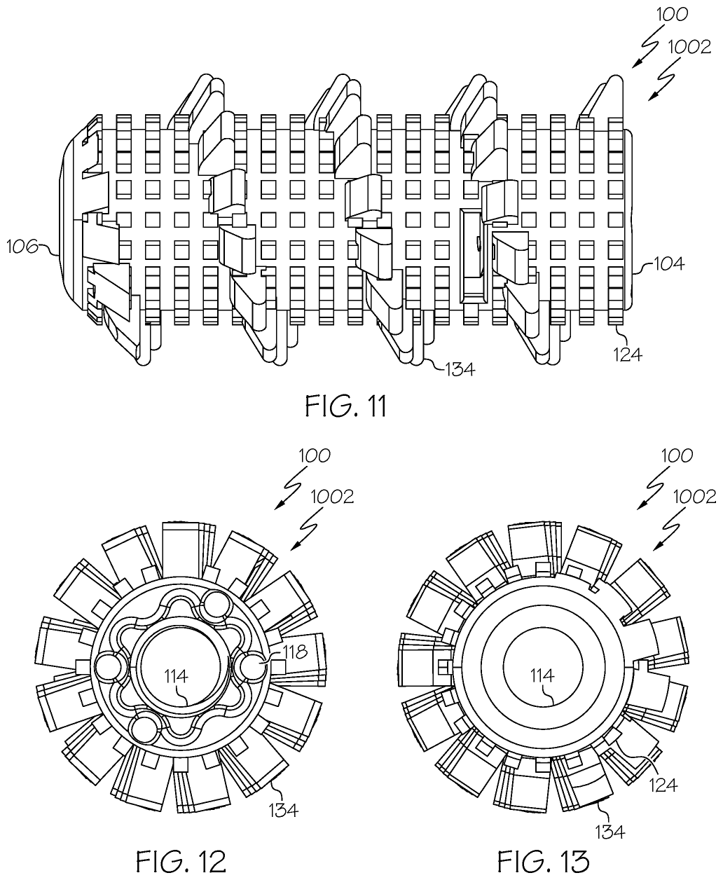

Considering additional features, FIGS. 8-14 illustrate a second embodiment 1002 of a hard-tissue implant 100. In accordance with this embodiment, the shaft 102 of the hard-tissue implant 100 comprises a top end aperture 112. The shaft 102 has an internal passage 114 extending axially with respect to the shaft 102, from the top end aperture 112. The internal passage 114 extends through the shaft 102, from the top end aperture 112, to a bottom end aperture 116 located at the bottom end 106 of the shaft 102. The hard-tissue implant 100 further comprises a tool-engaging portion 140, comprising a thread 142 located in the internal passage 114 of the shaft 102. The shaft 102 also comprises channels 118 through which sutures can be passed.

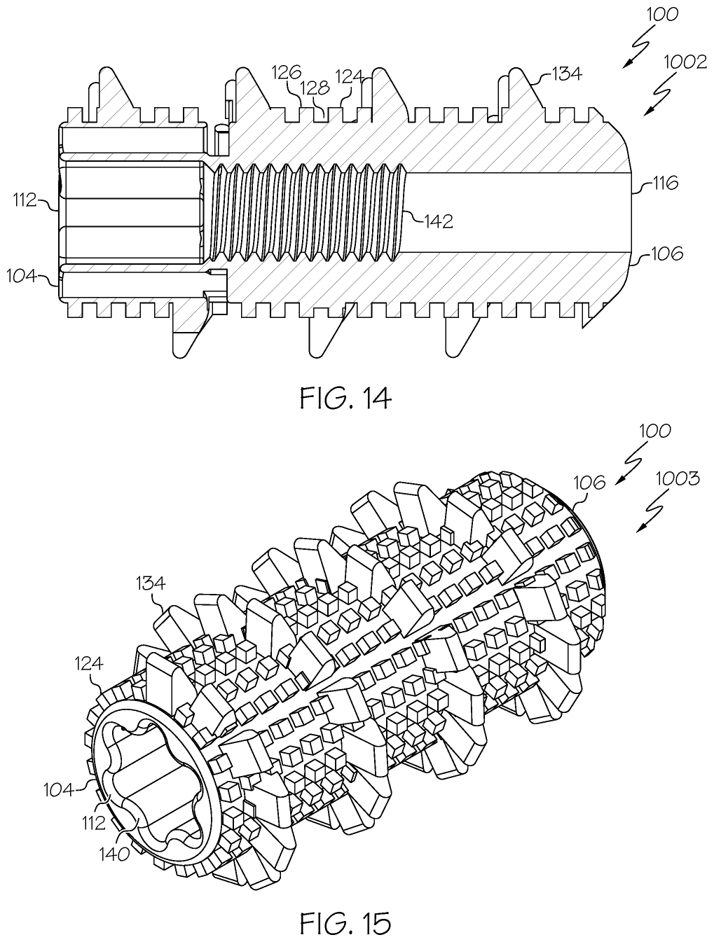

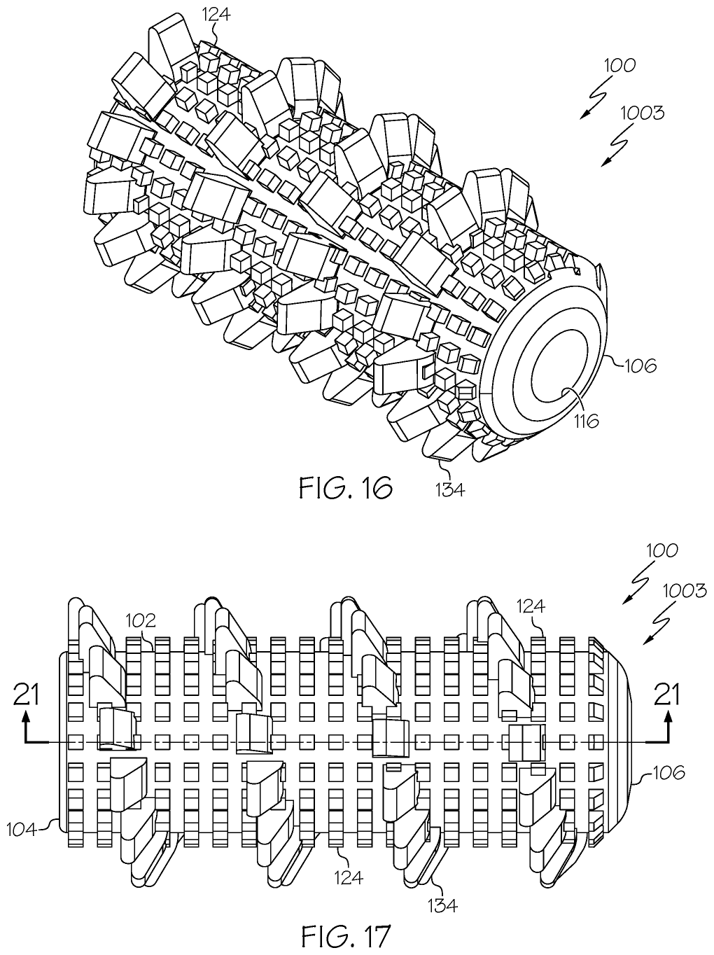

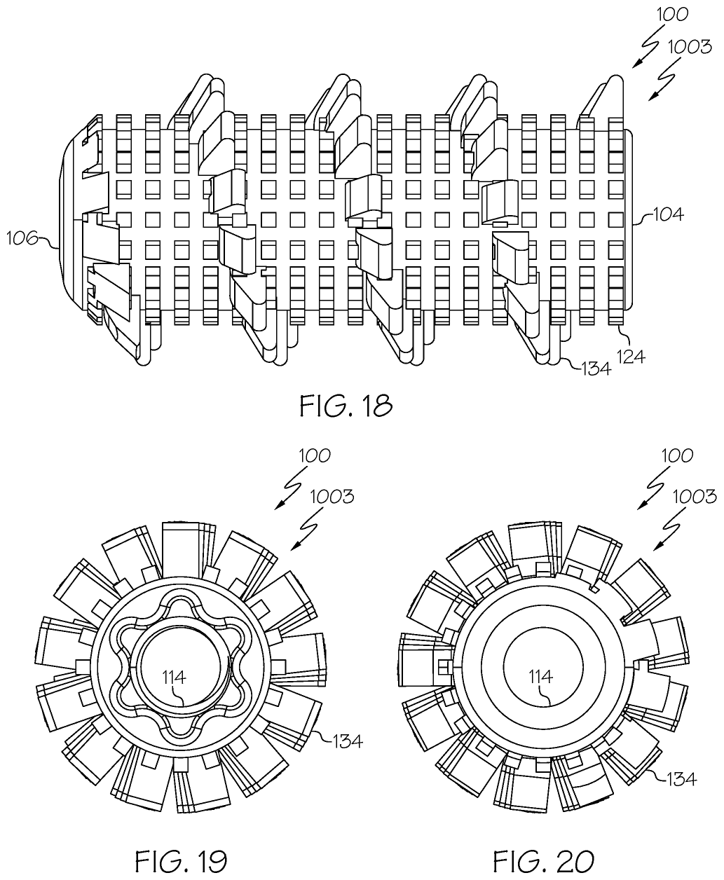

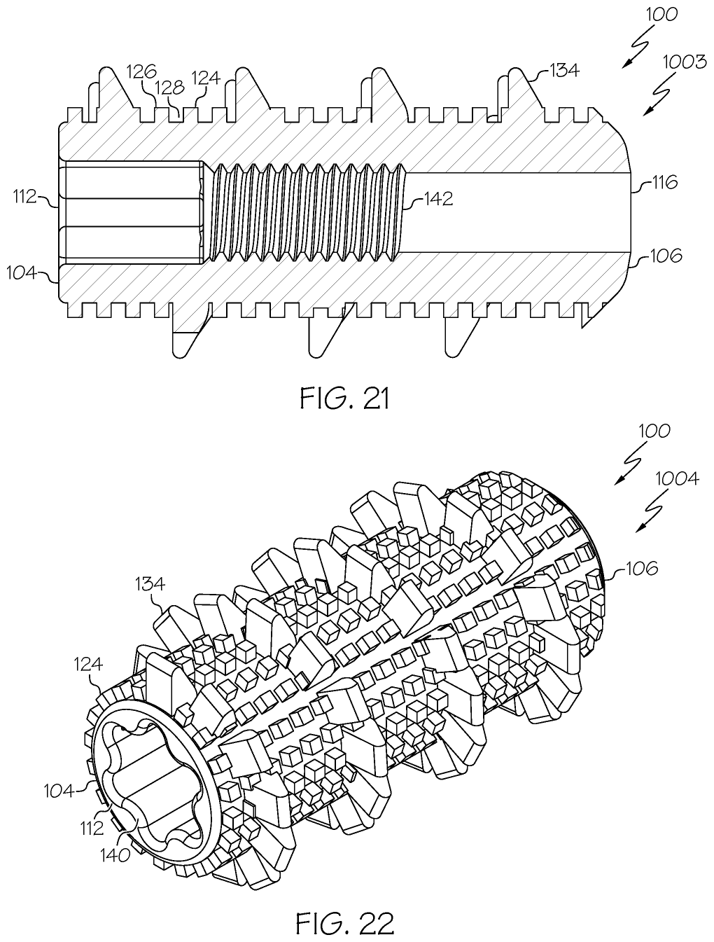

Considering further additional features, FIGS. 15-21 illustrate a third embodiment 1003 of a hard-tissue implant 100. In accordance with this embodiment, the shaft 102 of the hard-tissue implant 100 comprises a top end aperture 112. The shaft 102 has an internal passage 114 extending axially with respect to the shaft 102, from the top end aperture 112. The internal passage 114 extends through the shaft 102, from the top end aperture 112, to a bottom end aperture 116 located at the bottom end 106 of the shaft 102. The hard-tissue implant 100 further comprises a tool-engaging portion 140, comprising a thread 142 located in the internal passage 114 of the shaft 102.

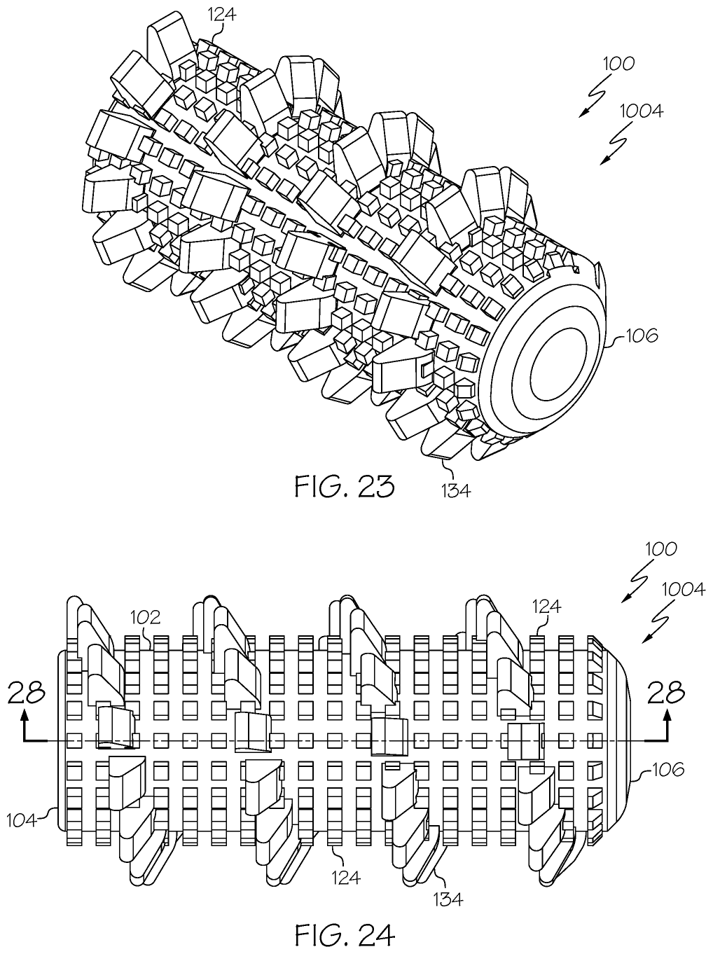

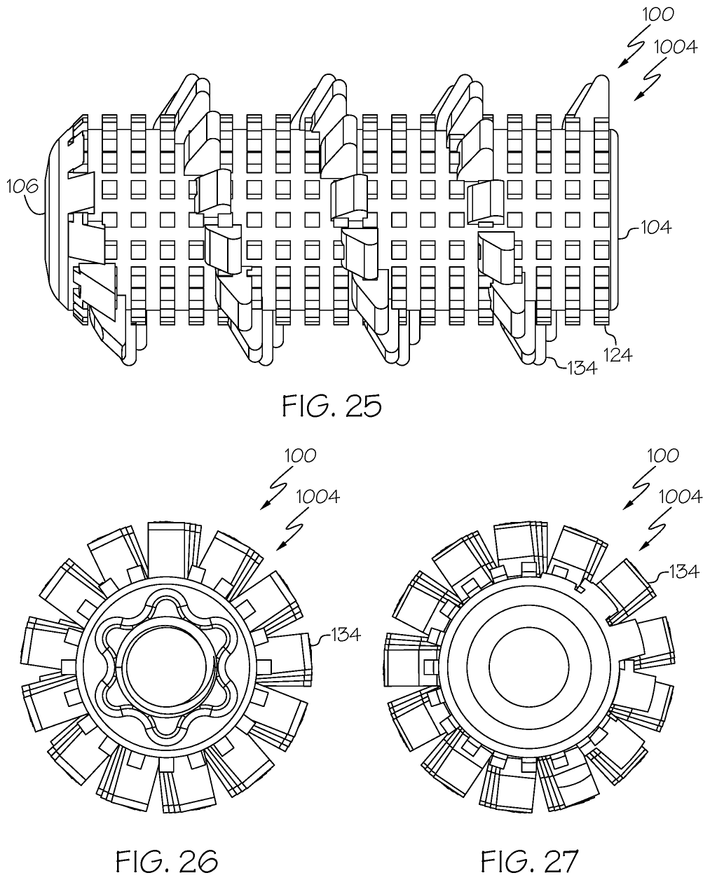

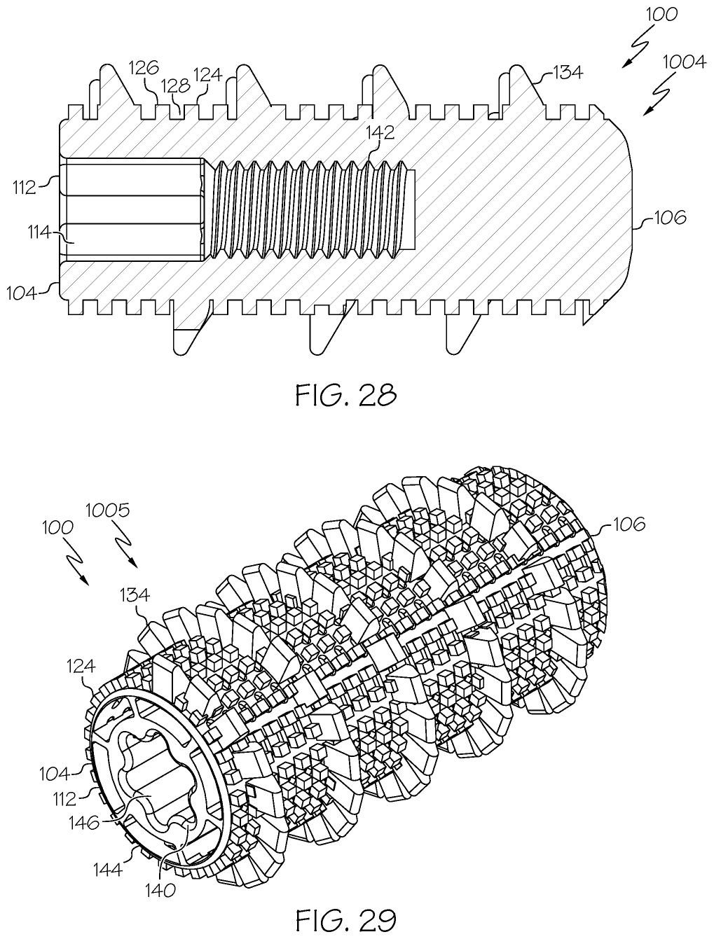

Considering further additional features, FIGS. 22-28 illustrate a fourth embodiment 1004 of a hard-tissue implant 100. In accordance with this embodiment, the shaft 102 of the hard-tissue implant 100 comprises a top end aperture 112. The shaft 102 has an internal passage 114 extending axially with respect to the shaft 102, from the top end aperture 112. The internal passage 114 extends through the shaft 102, from the top end aperture 112, and ends within the shaft 102. The hard-tissue implant 100 further comprises a tool-engaging portion 140, comprising a thread 142 located in the internal passage 114 of the shaft 102.

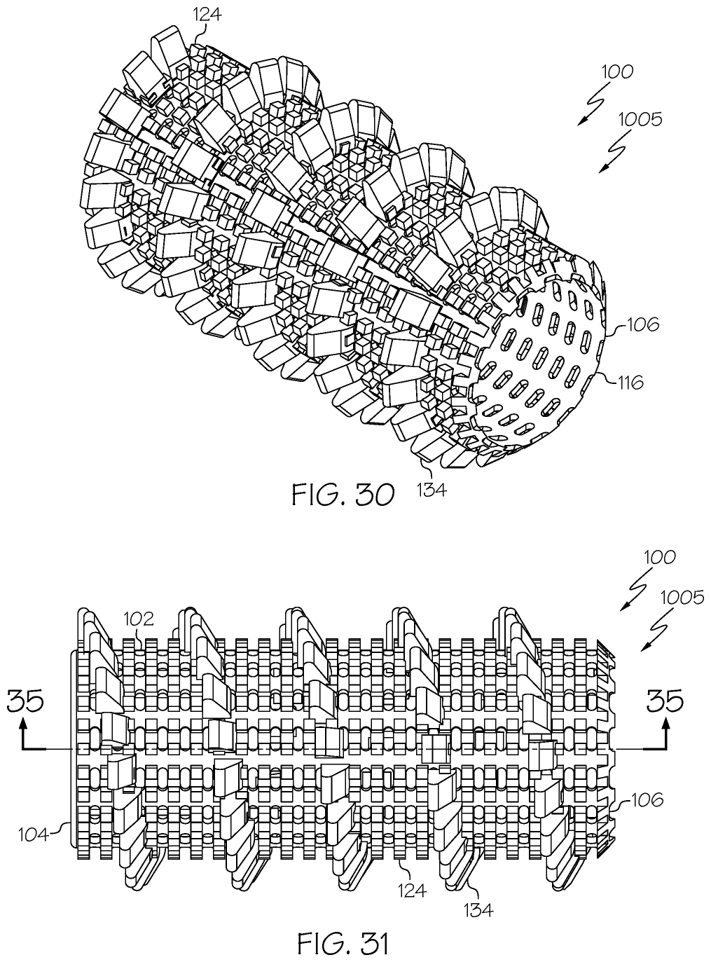

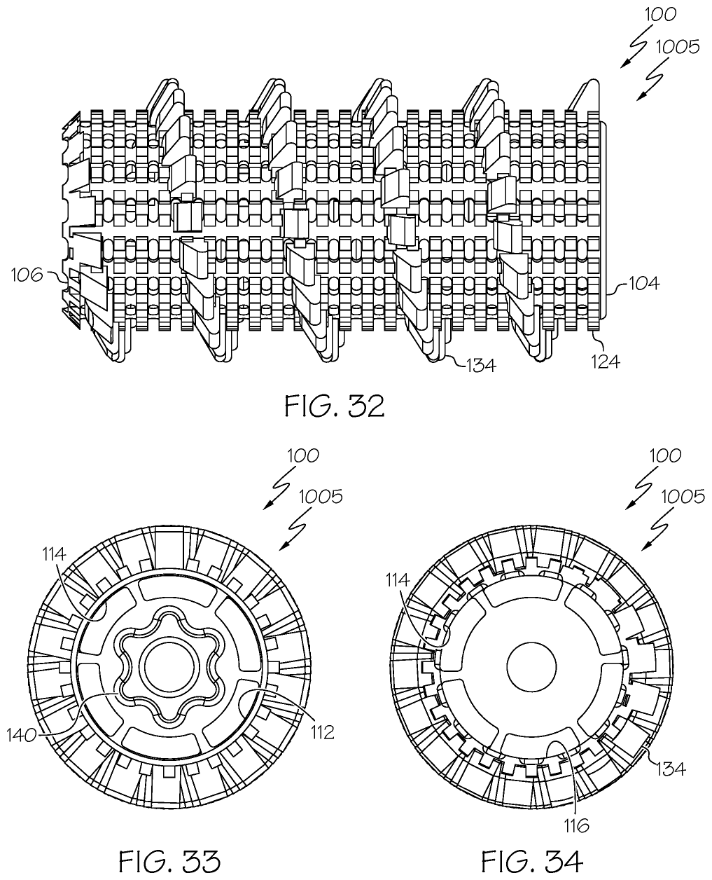

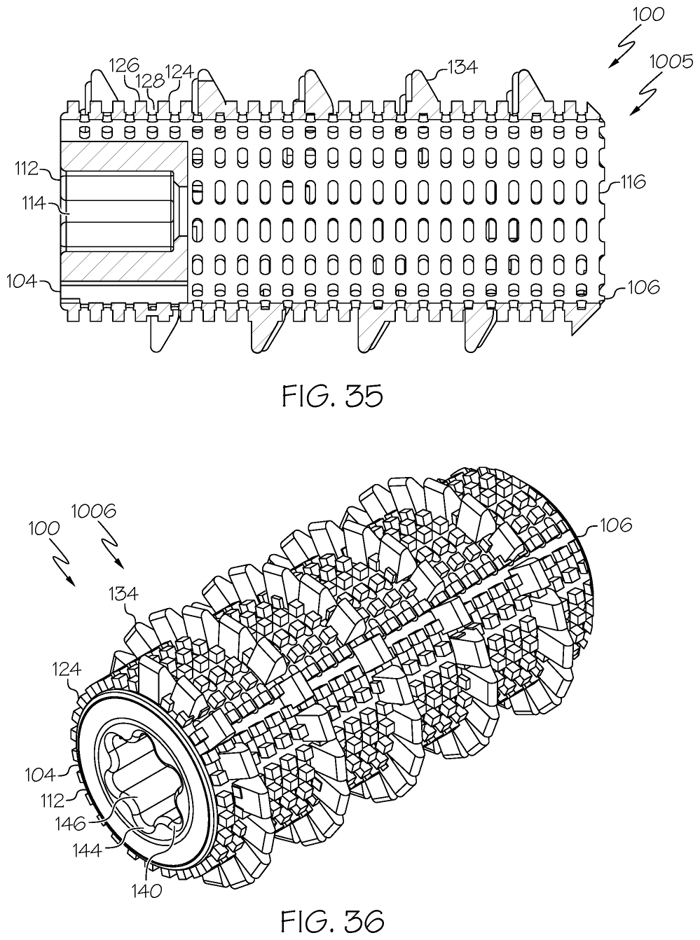

Considering further additional features, FIGS. 29-35 illustrate a fifth embodiment 1005 of a hard-tissue implant 100. In accordance with this embodiment, the shaft 102 of the hard-tissue implant 100 comprises a top end aperture 112. The shaft 102 has an internal passage 114 extending axially with respect to the shaft 102, from the top end aperture 112. The internal passage 114 extends through the shaft 102, from the top end aperture 112, to a bottom end aperture 116 located at the bottom end 106 of the shaft 102. The internal passage 114 is sufficiently extensive that the shaft 102 is essentially hollow. The shaft 102 is fenestrated. The hard-tissue implant 100 further comprises a tool-engaging portion 140, comprising a head 144 including a recess 146 located at the top end 104 of the shaft 102.

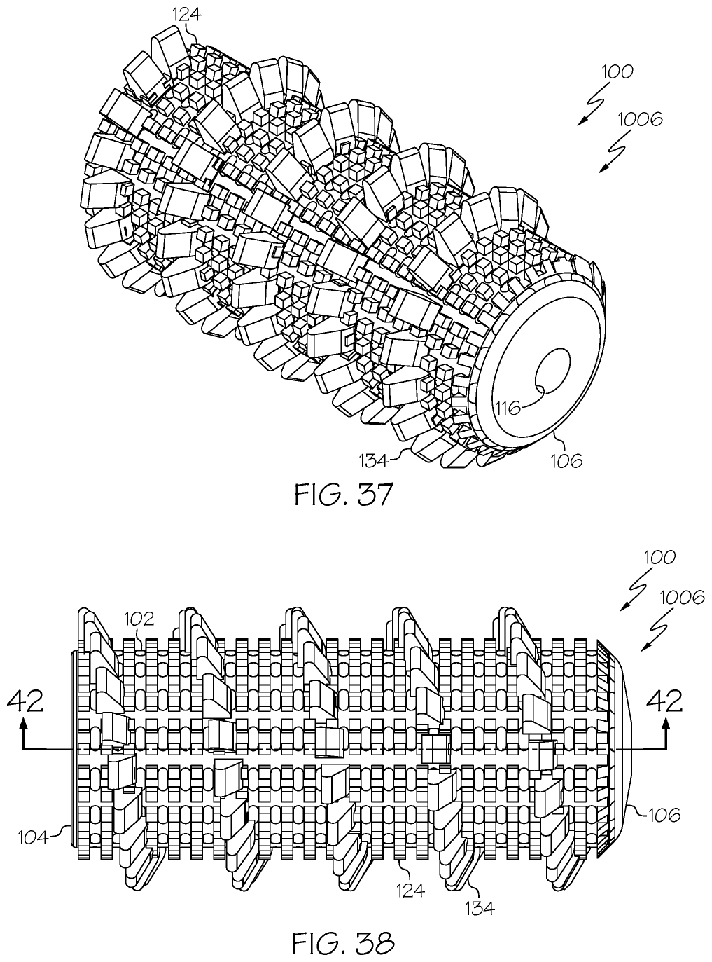

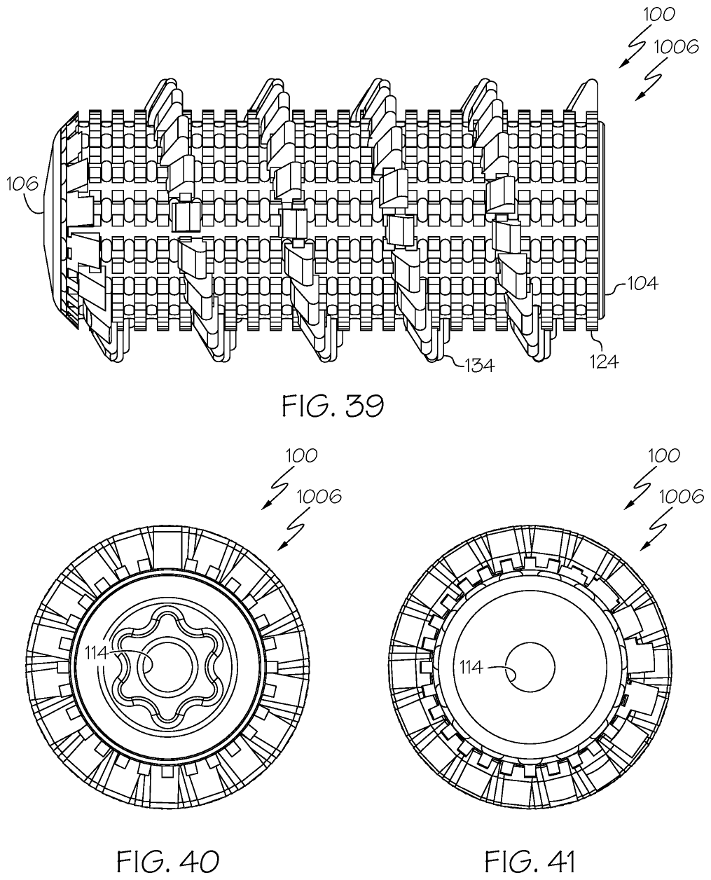

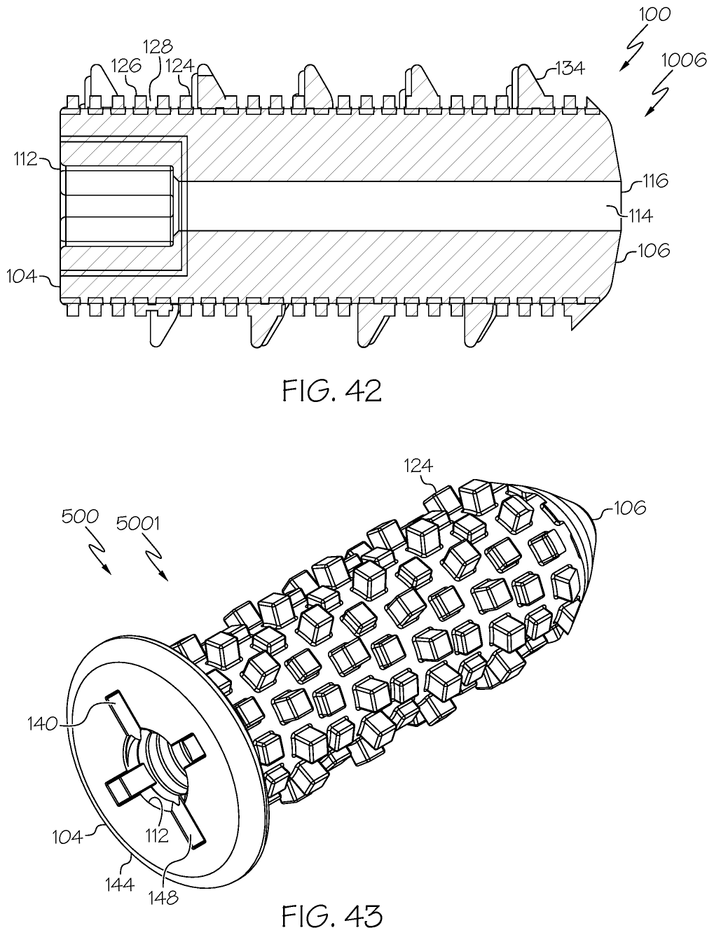

Considering further additional features, FIGS. 36-42 illustrate a sixth embodiment 1006 of a hard-tissue implant 100. In accordance with this embodiment, the shaft 102 of the hard-tissue implant 100 comprises a top end aperture 112. The shaft 102 has an internal passage 114 extending axially with respect to the shaft 102, from the top end aperture 112. The internal passage 114 extends through the shaft 102, from the top end aperture 112, to a bottom end aperture 116 located at the bottom end 106 of the shaft 102. The shaft 102 of the hard-tissue implant 100 includes pores. The hard-tissue implant 100 further comprises a tool-engaging portion 140, comprising a head 144 including a recess 146 located at the top end 104 of the shaft 102. In other examples of this embodiment, along with having pores, the shaft 102 can be hollow and/or fenestrated.

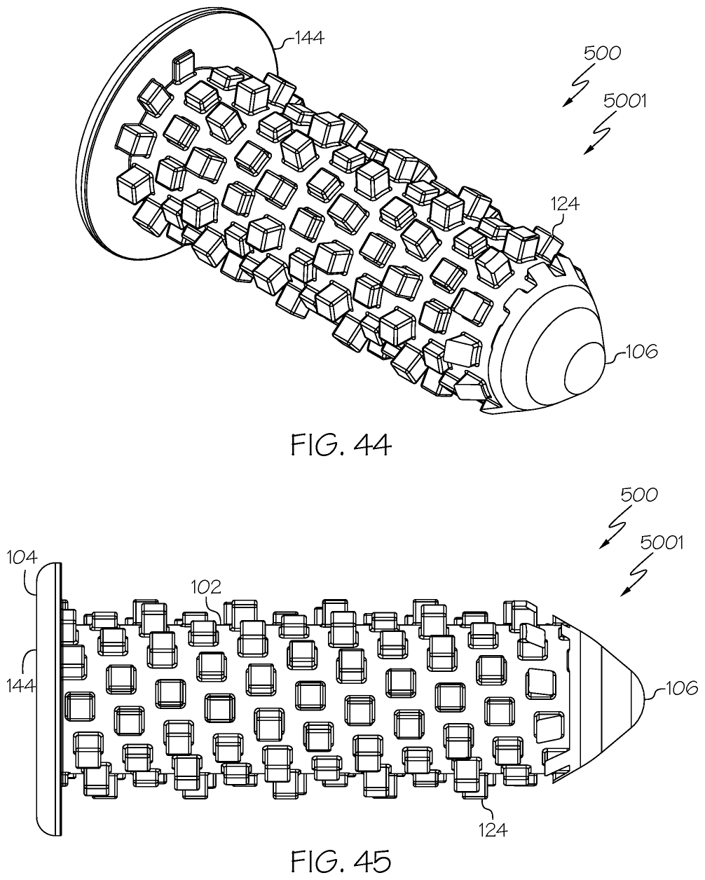

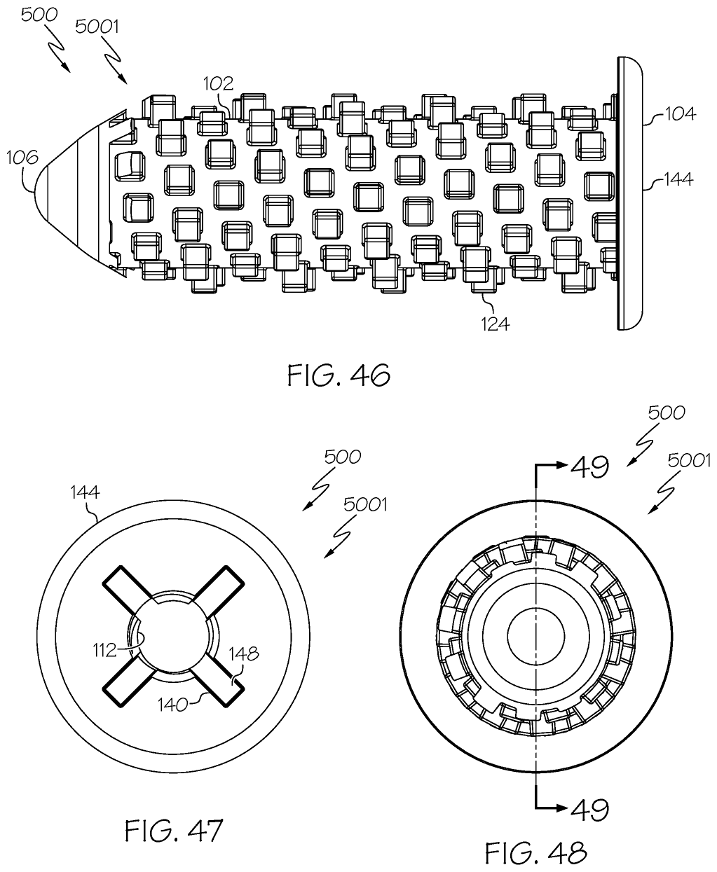

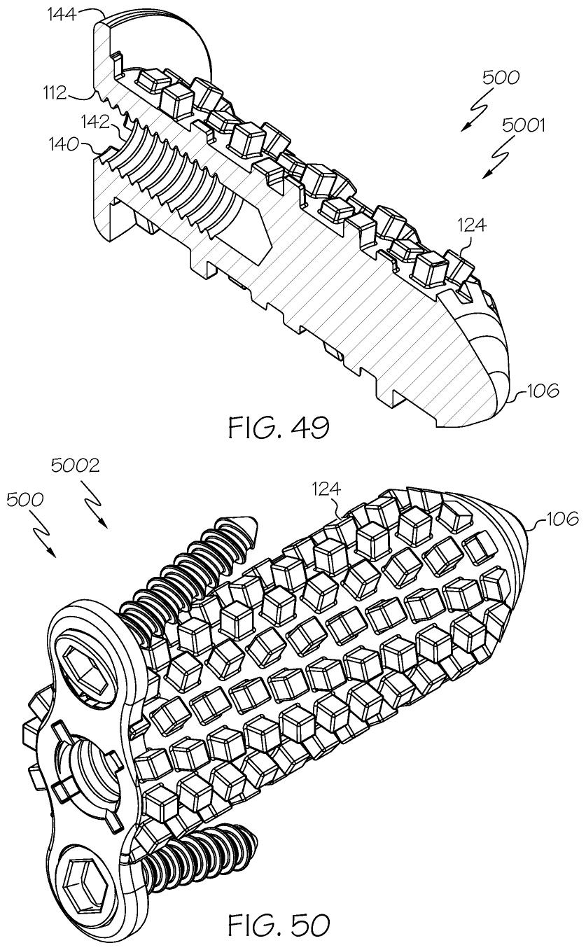

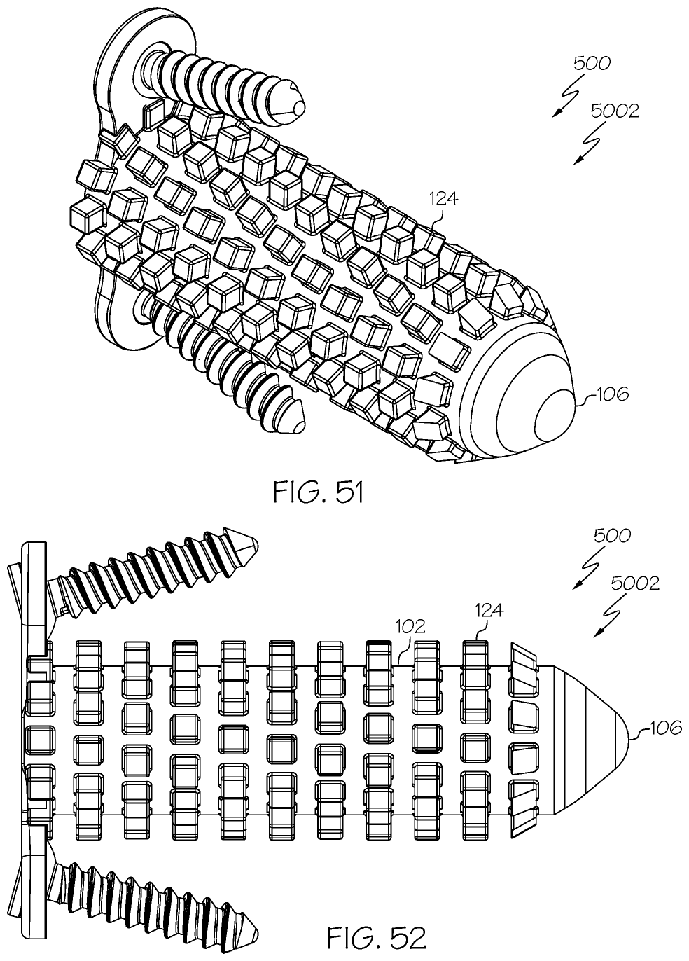

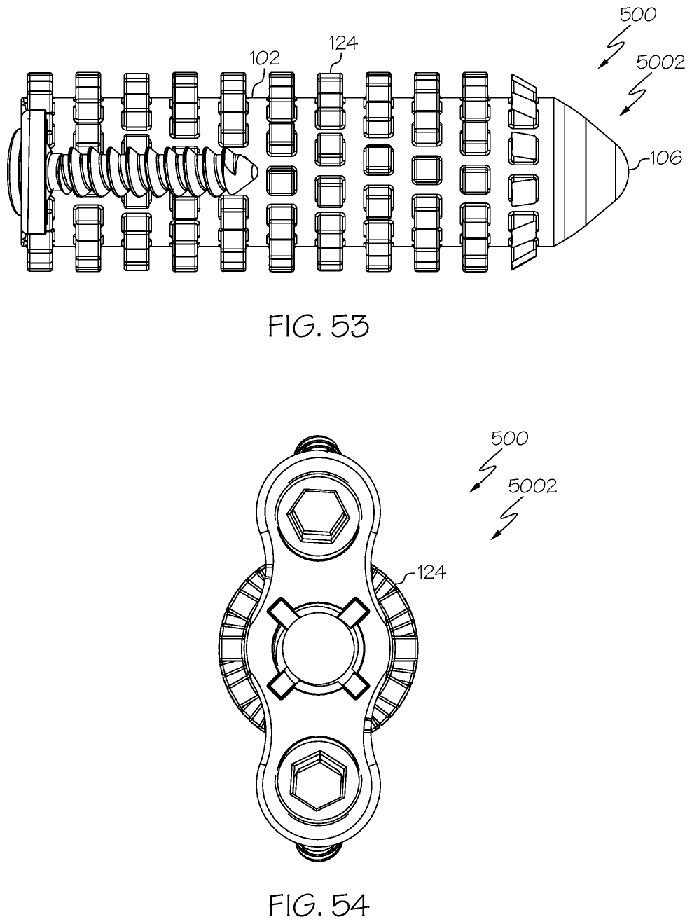

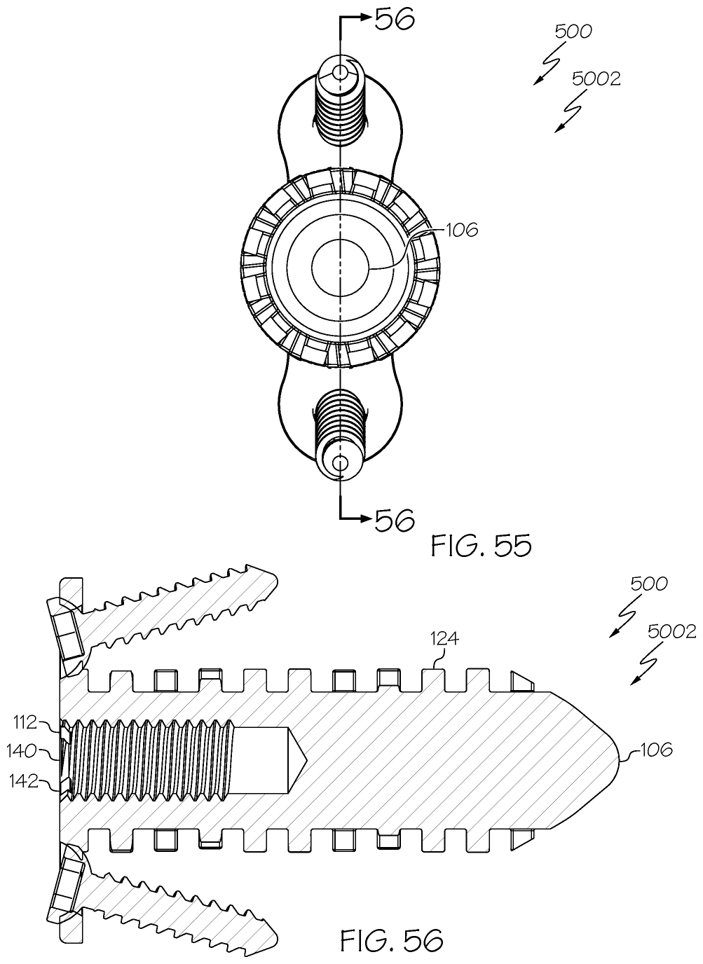

Also disclosed is another hard-tissue implant 500. The other hard-tissue implant 500 is like the hard-tissue implant 100 as disclosed above, except that the other hard-tissue implant 500 does not include a thread. FIGS. 43-49 illustrate a first embodiment 5001 of the other hard-tissue implant 500. FIGS. 50-56 illustrate a second embodiment 5002 of the other hard-tissue implant 500.

The implant 100 can be made by fabrication methods such as laser cutting, injection molding, or 3D printing, among others. The implant 500 also can be made by fabrication methods such as laser cutting, injection molding, or 3D printing, among others.

Methods of Using the Hard-Tissue Implants

Methods will now be described for use of the hard-tissue implant 100 for fusion of two or more bones in an individual in need thereof. The hard-tissue implant 100 is as described above. The method can be used for fusion of two bones, three bones, four bones, five bones, or a higher number of bones.

The method includes a step of (1) preparing a hole in at least a first bone and a second bone of the individual. In some examples the preparing of the hole comprises drilling a hole in at least the first bone and the second bone. In some examples, the hole is drilled such that the hole has an inner diameter, not including threads added by tapping, slightly smaller than an outer diameter 150 of the hard-tissue implant 100 including the pillars 124. Thus, in some examples, the hard-tissue implant 100 has an outer diameter 150 between distal ends 126 of pillars 124 at a widest portion of the shaft 102, and the preparing of the hole comprises preparing the hole to have a hole diameter that is smaller than the diameter 150. Moreover, in some examples the preparing of the hole comprises tapping the hole with a tapping device. In some of these examples the hole is tapped to have threads that have an inner diameter slightly smaller than an outer diameter 152 of the hard-tissue implant 100 including the thread segments 134.

The method also includes a step of (2) rotationally driving the hard-tissue implant 100 into the hole, such that the hard-tissue implant 100 contacts at least the first bone and the second bone and limits motion therebetween. For examples in which the hole is drilled such that the hole has an inner diameter, not including threads added by tapping, slightly smaller than an outer diameter 150 of the hard-tissue implant 100 including the pillars 124, rotationally driving the hard-tissue implant 100 into the hole can result in driving of the pillars 124 into bone defining the hole. For example, the hard-tissue implant 100 can be driven into the hole such that the pillars 124 penetrate bone defining the hole to a depth of, for example, 100 to 2,000 200 to 900 300 to 800 or 400 to 600 μm. Also for example, the hard-tissue implant 100 can be driven into the hole such that pillars 124 penetrate bone defining the hole to a depth, relative to the height of the pillars 124, of for example 25%, 50%, 75%, and 100% of the height of the pillars 124. Similarly, for examples in which the hole is tapped to have threads that have an inner diameter slightly smaller than an outer diameter 152 of the hard-tissue implant 100 including the thread segments 134, rotationally driving the hard-tissue implant 100 into the hole can result in driving the thread segments 134 into bone of the tapped thread.

In some examples the driving of the hard-tissue implant 100 into the hole results in compression of at least the first bone and the second bone.

In some examples the first bone comprises one or more metatarsal bones and the second bone comprises one or more cuneiform bones. In some of these examples, the driving of the hard-tissue implant 100 into the hole limits motion of first and second metatarsals and medial and intermediate cuneiforms of the individual, corresponding to a 1-2 TMT fusion. Also, in some of these examples the driving of the hard-tissue implant 100 into the hole limits motion of second and third metatarsals and intermediate and lateral cuneiforms of the individual, corresponding to a 2-3 TMT fusion.

In some examples the first bone comprises one or more navicular bones and the second bone comprises one or more cuneiform bones. In some of these examples the driving of the hard-tissue implant 100 into the hole limits motion of first and second navicular and medial and intermediate cuneiforms of the individual, corresponding to a 1-2 NC fusion. Also, in some of these examples the driving of the hard-tissue implant 100 into the hole limits motion of second and third navicular and intermediate and lateral cuneiforms of the individual, corresponding to a 2-3 NC fusion.

In some examples the first bone comprises talus and the second bone comprises calcaneus. In these examples, the driving of the hard-tissue implant 100 into the hole limits motion of the talus and the calcaneus.

In some examples the first bone comprises a proximal phalanx of hand and the second bone comprises a middle phalanx of hand. In these examples, the driving of the hard-tissue implant 100 into the hole limits motion of the proximal and middle phalanges.

In some examples the first bone comprises a middle phalanx of hand and the second bone comprises a distal phalanx of hand. In these examples, the driving of the hard-tissue implant 100 into the hole limits motion of the middle and distal phalanges.

In some examples the first bone comprises a first wrist bone and the second bone comprises a second wrist bone. In these examples, the driving of the hard-tissue implant 100 into the hole limits motion of the wrist bones.

In some examples the method further comprises one or more steps of (0) realigning the two more bones and/or removing cartilage between the two or more bones before the step of (1) preparing a hole in at least the first bone and the second bone of the individual. The additional one or more steps can promote fixation of the two or more bones.

In some examples the method further comprises: (1) preparing another hole in at least the first bone and the second bone of the individual; and (2) rotationally driving another of the hard-tissue implant 100 into the hole, such that the other hard-tissue implant 100 also contacts at least the first bone and the second bone and limits motion therebetween. Thus, in some examples two, three, or more of the hard-tissue implants 100 are used for fusion of the two or more bones.

Implantation of the hard-tissue implant 100 can accomplish fusion of the two or more bones without use of screws or plating mechanisms. As noted above, the pillars 124 may be rotationally driven into the hard-tissue during implantation, potentially eliminating micro-motion and migration of the hard-tissue implant 100 over time, accommodating torque, and/or eliminating the need for adhesives such as cement or grout to hold the hard-tissue implant 100 in place. Accordingly, in some examples the method does not comprise using plates or screws to limit motion between the first bone and the second bone. This can minimize the number and profiles of implants used in the method in an individual while still eliminating micro-motion and migration of the hard-tissue implant 100 over time.

Also, implantation of the hard-tissue implant 100 can accomplish fusion of the two or more bones without use of adhesives, e.g. cement or grout. Accordingly, in some examples the method does not comprise using adhesives. This can simplify the method while still eliminating micro-motion and migration of the hard-tissue implant 100 over time.

In some examples additional hard tissue can be added to the surface 120 of the shaft 102 and/or the pillars 124 and/or in an internal passage 114 and/or a hollow center of the hard-tissue implant 100 prior to implanting. For example, shavings of hard-tissue of a patient, generated during preparation work including sawing or drilling of hard tissue of the patient, can be added. This may promote growth of hard tissue into the slots 128 and/or around the hard-tissue implant 100 following implantation.

Also in some examples additional compositions can be added to the surface 120 of the shaft 102 and/or the pillars 124 and/or in an internal passage 114 and/or a hollow center of the hard-tissue implant 100 prior to implanting. Such compositions include, for example, blood, one or more antibiotics, one or more osteogenic compounds, bone marrow aspirate, and/or surface chemistry for inducing early bone ingrowth. For example, the surface 120 and/or the pillars 124 can be coated with one or more such compositions, with the pillars 124 retaining the compositions during implantation. This also may promote growth of tissue into the slots 128 and/or around the hard-tissue implant 100 following implantation.

Standard approaches for rotationally driving implants can be used in the methods disclosed here.

The method can be applied to the embodiments and examples of the hard-tissue implant 100 as disclosed above. The ratio of (i) the sum of the volumes of the slots 128 and the thread gaps 136 to (ii) the sum of the volumes of the pillars 124 and the thread segments 134 and the volumes of the slots 128 and the thread gaps 136 can be determined as discussed above.

The method also can be applied to the embodiments of the implant 500 as disclosed above.

It will be apparent to those skilled in the art that various modifications and variations can be made without departing from the spirit and scope of the claimed invention.

What is claimed is:

1. A hard-tissue implant comprising:
(a) a shaft having a top end and a bottom end, the shaft extending between the top end and the bottom end;
(b) a surface of the shaft extending from the top end to the bottom end;
(c) pillars for contacting a hard tissue, the pillars being distributed on the surface across an area of at least 50 mm², and extending distally therefrom, and each pillar being integral to the shaft, having a distal end, having a transverse area of (100×100) to (2,000×2,000) μm², and having a height of 100 to 2,000 μm;
(d) slots to be occupied by the hard tissue, the slots being defined by the pillars and each slot having a width of 100 to 2,000 μm as measured along the shortest distance between adjacent pillars; and
(e) a thread disposed helically along the shaft, extending radially from the shaft, and having a plurality of grooves oriented transversely with respect to the thread that define a series of thread segments and thread gaps along the thread; wherein:
the implant has a Young's modulus of elasticity of at least 3 GPa and a ratio of (i) the sum of the volumes of the slots and the thread gaps to (ii) the sum of the volumes of the pillars and the thread segments and the volumes of the slots and the thread gaps of 0.40:1 to 0.90:1.

2. The implant of claim 1, wherein the transverse area of each pillar is (250×250) μm² to (1,000×1,000) μm².

3. The implant of claim 1, wherein the height of each pillar is 200 to 900 μm.

4. The implant of claim 1, wherein the width of each slot is 200 to 1,000 μm.

5. The implant of claim 1, wherein the thread has a thread height of 100 μm to 5,000 μm.

6. The implant of claim 1, wherein the thread segments have a thread segment width, measured as an arcuate length with respect to the shaft, of 100 μm to 5,000 μm.

7. The implant of claim 1, wherein the thread gaps have a thread gap width, measured as an arcuate length with respect to the shaft, of 100 μm to 5,000 μm.

8. The implant of claim 1, wherein the shaft has a shaft diameter at a widest portion of the shaft and a shaft length from the top end to the bottom end, and the implant has a ratio of the shaft length to the shaft diameter of 2.0 to 10.

9. The implant of claim 1, wherein the shaft has a shaft diameter of 3 to 20 mm at a widest portion of the shaft.

10. The implant of claim 1, wherein the shaft has a shaft length of 6 to 40 mm from the top end to the bottom end.

11. A method of use of the implant of claim 1 for fusion of two or more bones in an individual in need thereof, the method comprising steps of:
(1) preparing a hole in at least a first bone and a second bone of the individual; and
(2) rotationally driving the implant into the hole, such that the implant contacts at least the first bone and the second bone and limits motion therebetween.

12. The method of claim 11, wherein the preparing of the hole comprises drilling a hole in at least the first bone and the second bone.

13. The method of claim 11, wherein the implant has an outer diameter between distal ends of pillars at a widest portion of the shaft, and the preparing of the hole comprises preparing the hole to have a hole diameter that is smaller than the outer diameter.

14. The method of claim 11, wherein the first bone comprises one or more metatarsal bones and the second bone comprises one or more cuneiform bones.

15. The method of claim 11, wherein the first bone comprises one or more navicular bones and the second bone comprises one or more cuneiform bones.

16. The method of claim 11, wherein the first bone comprises talus and the second bone comprises calcaneus.

17. The method of claim 11, wherein the first bone comprises a proximal phalanx of hand and the second bone comprises a middle phalanx of hand.

18. The method of claim 11, wherein the first bone comprises a middle phalanx of hand and the second bone comprises a distal phalanx of hand.

19. The method of claim 11, wherein the first bone comprises a first wrist bone and the second bone comprises a second wrist bone.

20. The method of claim 11, wherein the method does not comprise using plates or screws to limit motion between the first bone and the second bone.
